SEQUENCE LISTING (34 sequences)

SEQ ID NO: 1 - DNA, 1226 bp, Oryza sativa Japonica
atgcaccctcacctgtaccagagccaccaccaccaccagcacgttgctcctccgaccaagcatggcggcggcggcgccggcgccggcggcgaggaggacgaccagtccatgcttctcctctcgctctggcctcccggccacagcagccaccaccaccaacaccaccaccatggtgcggcctcgtcgtcggcgtgctcgtctgcgtccatctccgccgccgctcactctggtggtggtggtggtggccatggcgccgccgccgccgccgcatcgtcatggttcaacccgagccactaccatggcggcggcggaggcggaggtgggttcttgttccaggagcagcaggagcccaacgtgagcatatcgctgtccatagcccctccctgcggcgtcagcggcggcaacgcggcggcgatggcggcggcaggcagcttcgccacctcgccgttgccgcagcagccggtggcgtcgtcggcggtggccacggcggcgggcggcgggcaggctcccagccagtactggatcccctccgccgccgagatcctcgtcggctccacccagttctcctgcgccgtctgcaacaagaccttcaaccgcttcaacaacatgcaggtaatcaattagcttctccaatcgccatggacgccgaccaactcgagctcatgtaattcttgatgcagatgcacatgtgggggcatgggtcgcagtacaggaaagggtcggagtcgctgaggggggcgatcacggtgggcggggcggcgccgccggcgtcgctgatgcggctgccgtgctactgctgcgcggaggggtgccggaacaacgtggagcacccgcgcgcccgcccgctcaaggacttccggacgctgcagacgcactaccggcggaagcacggcgcccgcccctacgcctgccgccgctgcggcaagcgcttcgccgtgcgcggcgactggcgcacccacgagaagaactgcggcaagctctggttctgcgtctgcggctccgacttcaagcacaagcgctccctcaaggaccacgtccgctccttcggcggcggccacgccccccacatcgtcgagtcttcctccaccggcgccggcggtggcggcggatgcggcggcgccggcgctgatgacgatgacgatgagtactacgacgacgacgacgacaccgtctcgctctcccgcgccaccagcgcgcacgacgacgccgacatgatcgtcgtctag

SEQ ID NO: 2 - DNA, 1158 bp, Oryza sativa Japonica
atgcaccctcacctgtaccagagccaccaccaccaccagcacgttgctcctccgaccaagcatggcggcggcggcgccggcgccggcggcgaggaggacgaccagtccatgcttctcctctcgctctggcctcccggccacagcagccaccaccaccaacaccaccaccatggtgcggcctcgtcgtcggcgtgctcgtctgcgtccatctccgccgccgctcactctggtggtggtggtggtggccatggcgccgccgccgccgccgcatcgtcatggttcaacccgagccactaccatggcggcggcggaggcggaggtgggttcttgttccaggagcagcaggagcccaacgtgagcatatcgctgtccatagcccctccctgcggcgtcagcggcggcaacgcggcggcgatggcggcggcaggcagcttcgccacctcgccgttgccgcagcagccggtggcgtcgtcggcggtggccacggcggcgggcggcgggcaggctcccagccagtactggatcccctccgccgccgagatcctcgtcggctccacccagttctcctgcgccgtctgcaacaagaccttcaaccgcttcaacaacatgcagatgcacatgtgggggcatgggtcgcagtacaggaaagggtcggagtcgctgaggggggcgatcacggtgggcggggcggcgccgccggcgtcgctgatgcggctgccgtgctactgctgcgcggaggggtgccggaacaacgtggagcacccgcgcgcccgcccgctcaaggacttccggacgctgcagacgcactaccggcggaagcacggcgcccgcccctacgcctgccgccgctgcggcaagcgcttcgccgtgcgcggcgactggcgcacccacgagaagaactgcggcaagctctggttctgcgtctgcggctccgacttcaagcacaagcgctccctcaaggaccacgtccgctccttcggcggcggccacgccccccacatcgtcgagtcttcctccaccggcgccggcggtggcggcggatgcggcggcgccggcgctgatgacgatgacgatgagtactacgacgacgacgacgacaccgtctcgctctcccgcgccaccagcgcgcacgacgacgccgacatgatcgtcgtctag

SEQ ID NO: 3 - protein, 385 aa, Oryza sativa Japonica
MHPHLYQSHHHHQHVAPPTKHGGGGAGAGGEEDDQSMLLLSLWPPGHSSHHHQHHHHGAASSSACSSASISAAAHSGGGGGGHGAAAAAASSWFNPSHYHGGGGGGGGFLFQEQQEPNVSISLSIAPPCGVSGGNAAAMAAAGSFATSPLPQQPVASSAVATAAGGGQAPSQYWIPSAAEILVGSTQFSCAVCNKTFNRFNNMQMHMWGHGSQYRKGSESLRGAITVGGAAPPASLMRLPCYCCAEGCRNNVEHPRARPLKDFRTLQTHYRRKHGARPYACRRCGKRFAVRGDWRTHEKNCGKLWFCVCGSDFKHKRSLKDHVRSFGGGHAPHIVESSSTGAGGGGGCGGAGADDDDDEYYDDDDDTVSLSRATSAHDDADMIVV

SEQ ID NO: 4 - DNA, 23 bp, Artificial Sequence
  sgRNA1
ttcaacccgagccactaccatgg

SEQ ID NO: 5 - DNA, 23 bp, Artificial Sequence
  sgRNA3
tggtggctctggtacaggtgagg

SEQ ID NO: 6 - DNA, 23 bp, Artificial Sequence
  sgRNA4
cgtacagtctagctaggtatagg

SEQ ID NO: 7 - DNA, 21 bp, Artificial Sequence
  microRNA targeting first exon of LOC_Os09g13680
ttatgctcacgttaggctcat

SEQ ID NO: 8 - DNA, 21 bp, Artificial Sequence
  microRNA targeting first exon of LOC_Os09g13680
ttatgctcacgttaggctcgt

SEQ ID NO: 9 - DNA, 21 bp, Artificial Sequence
  microRNA targeting first exon of LOC_Os09g13680
tacaagaacccacatccgcat

SEQ ID NO: 10 - DNA, 40 bp, Artificial Sequence
  Oligos for amiRNA “TTATGCTCACGTTAGGCTCAT” miR-s
gattatgctcacgttaggctcattctctcttttgtattcc

SEQ ID NO: 11 - DNA, 40 bp, Artificial Sequence
  Oligos for amiRNA “TTATGCTCACGTTAGGCTCAT” miR-a
gaatgagcctaacgtgagcataatcaaagagaatcaatga

SEQ ID NO: 12 - DNA, 40 bp, Artificial Sequence
  Oligos for amiRNA “TTATGCTCACGTTAGGCTCAT” miR*s
gaataagcctaacgtcagcatattcacaggtcgtgatatg

SEQ ID NO: 13 - DNA, 40 bp, Artificial Sequence
  Oligos for amiRNA “TTATGCTCACGTTAGGCTCAT” miR*a
gaatatgctgacgttaggcttattctacatatatattcct

SEQ ID NO: 14 - DNA, 40 bp, Artificial Sequence
  Oligos for amiRNA “TTATGCTCACGTTAGGCTCGT” miR-s
agttatgctcacgttaggctcgtcaggagattcagtttga

SEQ ID NO: 15 - DNA, 40 bp, Artificial Sequence
  Oligos for amiRNA “TTATGCTCACGTTAGGCTCGT” miR-a
tgacgagcctaacgtgagcataactgctgctgctacagcc

SEQ ID NO: 16 - DNA, 40 bp, Artificial Sequence
  Oligos for amiRNA “TTATGCTCACGTTAGGCTCGT” miR*s
ctacgaggctatcgtgagcataattcctgctgctaggctg

SEQ ID NO: 17 - DNA, 40 bp, Artificial Sequence
  Oligos for amiRNA “TTATGCTCACGTTAGGCTCGT” miR*a
aattatgctcacgatagcctcgtagagaggcaaaagtgaa

SEQ ID NO: 18 - DNA, 40 bp, Artificial Sequence
  Oligos for amiRNA “TACAAGAACCCACATCCGCAT” miR-s
agtacaagaacccacatccgcatcaggagattcagtttga

SEQ ID NO: 19 - DNA, 40 bp, Artificial Sequence
  Oligos for amiRNA “TACAAGAACCCACATCCGCAT” miR-a
tgatgcggatgtgggttcttgtactgctgctgctacagcc

SEQ ID NO: 20 - DNA, 40 bp, Artificial Sequence
  Oligos for amiRNA “TACAAGAACCCACATCCGCAT” miR*s
ctatgcgcatgagggttcttgtattcctgctgctaggctg

SEQ ID NO: 21 - DNA, 23 bp, Artificial Sequence
  sgRNA74
cttgttgctggggggaacggcgg

SEQ ID NO: 22 - DNA, 23 bp, Artificial Sequence
  sgRNA153
agcatgccgagtaggatgagtgg

SEQ ID NO: 23 - DNA, 20 bp, Artificial Sequence
  Forward Primer
caaccaagctctgatagagt

SEQ ID NO: 24 - DNA, 19 bp, Artificial Sequence
  Reverse Primer
gaagaatctcgtgctttca

SEQ ID NO: 25 - DNA, 20 bp, Artificial Sequence
  Primer SvDOT1_sg65-F
cacttgtttctcccctccct

SEQ ID NO: 26 - DNA, 20 bp, Artificial Sequence
  Primer SvDOT1_sg153-R
tgtagtgactctggtggtgg

SEQ ID NO: 27 - protein, 385 aa, Artificial Sequence; identical to SEQ ID NO: 3
  Protein encoded by OsDOT1

SEQ ID NO: 28 - protein, 355 aa, Setaria viridis
MQHSYRAMHPYTHPYQHHAASAVPPSNKQHGDDDDDQSMLLLSLWPPGHHHHSSYSACSSPSIVSTTTSMSMAAATAAASNPWLSSHHQSHYNGGGGSSSSHSSFLFHEQAEPDVSISLSIAPPCGSAGFATAPAAPSTVTTTVAPGGGQQVPSQYWIPSAAEILVGSTQFSCAVCNKTFNRFNNMQMHMWGHGSQYRKGSESLRGAITVGTAPPASLMRLPCYCCAEGCRNNIEHPRARPLKDFRTLQTHYRRKHGARPYACRRCGKRFAVRGDWRTHEKNCGRLWFCVCGSDFKHKRSLKDHVRSFGGGHAPHIVESVAGVGGADDDEEDDDEHDDADLQMVDDNGGSDMVVV

SEQ ID NO: 29 - protein, 356 aa, Setaria viridis
MQHSYRAMHPYTHPYQHHAASAVPPSNKQHGDDDDDQSMLLLSLWPPGHHHHSILLGMLLPLHRLHHHLHVHGRRDRRRFQPLAQQPPPESLQRRRREQQQPQQLPVPRAGGARREHIALHRPAVRQRGLRHRPGGAVDGDDDRGAGRRPAGAQPVLDPLRRRDPRRLHAVLLRRLQQDLQPLQQHADAHVGPRLAVPQGLRVPARRDHGGHGAAGVADAAAVLLLRRGVPEQHRAPACSPPQGLPDAADALPPEARRAAVRVPAVREAVRRARRLAHPREELRQALVLRLRFRLQAQALAQGPRPLLRRGACAAHRRVRRRRRRRRRRRGGRRRTRRRRPADGGRQRRLRHGRRL

SEQ ID NO: 30 - protein, 354 aa, Setaria viridis
  MISC_FEATURE (54)..(54): “S” or “I”, predominantly I
  MISC_FEATURE (55)..(55): “Y” or “L”, predominantly L
  MISC_FEATURE (56)..(56): “S” or “L”, predominantly L
  MISC_FEATURE (57)..(57): “A” or “G”, predominantly A
  MISC_FEATURE (58)..(58): “C” or “M”, predominantly C
  MISC_FEATURE (59)..(59): “S” or “L”, predominantly L
  MISC_FEATURE (60)..(60): “S” or “L”, predominantly L
  MISC_FEATURE (62)..(62): “S” or “L”, predominantly L
  MISC_FEATURE (63)..(63): “I” or “H”, predominantly H
  MISC_FEATURE (64)..(64): “V” or “R”, predominantly R
  MISC_FEATURE (65)..(65): “S” or “L”, predominantly L
  MISC_FEATURE (66)..(66): “T” or “H”, predominantly H
  MISC_FEATURE (67)..(67): “T” or “H”, predominantly H
  MISC_FEATURE (68)..(68): “T” or “H”, predominantly H
  MISC_FEATURE (69)..(69): “S” or “L”, predominantly L
  MISC_FEATURE (70)..(70): “M” or “H”, predominantly H
  MISC_FEATURE (71)..(71): “S” or “V”, predominantly S
  MISC_FEATURE (72)..(72): “M” or “H”, predominantly H
  MISC_FEATURE (73)..(73): “A” or “G”, predominantly A
  MISC_FEATURE (74)..(74): “A” or “R”, predominantly A
  MISC_FEATURE (75)..(75): “A” or “R”, predominantly A
  MISC_FEATURE (76)..(76): “T” or “D”, predominantly D
  MISC_FEATURE (77)..(77): “A” or “R”, predominantly A
  MISC_FEATURE (78)..(78): “A” or “R”, predominantly A
  MISC_FEATURE (79)..(79): “A” or “R”, predominantly A
  MISC_FEATURE (80)..(80): “S” or “F”, predominantly F
  MISC_FEATURE (81)..(81): “N” or “Q”
  MISC_FEATURE (83)..(83): “W” or “L”, predominantly L
  MISC_FEATURE (84)..(84): “L” or “A”, predominantly A
  MISC_FEATURE (85)..(85): “S” or “Q”, predominantly Q
  MISC_FEATURE (86)..(86): “S” or “Q”, predominantly Q
  MISC_FEATURE (87)..(87): “H” or “P”, predominantly H
  MISC_FEATURE (88)..(88): “H” or “P”, predominantly H
  MISC_FEATURE (89)..(89): “Q” or “P”, predominantly Q
  MISC_FEATURE (90)..(90): “S” or “E”, predominantly E
  MISC_FEATURE (91)..(91): “H” or “S”, predominantly H
  MISC_FEATURE (92)..(92): “Y” or “L”, predominantly L
  MISC_FEATURE (93)..(93): “N” or “Q”
  MISC_FEATURE (94)..(94): “G” or “R”, predominantly R
  MISC_FEATURE (95)..(95): “G” or “R”, predominantly R
  MISC_FEATURE (96)..(96): “G” or “R”, predominantly R
  MISC_FEATURE (97)..(97): “G” or “R”, predominantly R
  MISC_FEATURE (98)..(98): “S” or “E”, predominantly E
  MISC_FEATURE (99)..(99): “S” or “Q”, predominantly Q
  MISC_FEATURE (100)..(100): “S” or “Q”, predominantly Q
  MISC_FEATURE (101)..(101): “S” or “Q”, predominantly Q
  MISC_FEATURE (102)..(102): “H” or “P”, predominantly H
  MISC_FEATURE (103)..(103): “S” or “Q”, predominantly Q
  MISC_FEATURE (104)..(104): “S” or “Q”, predominantly Q
  MISC_FEATURE (105)..(105): “F” or “L”, predominantly L
  MISC_FEATURE (106)..(106): “L” or “P”, predominantly L
  MISC_FEATURE (107)..(107): “F” or “V”, predominantly F
  MISC_FEATURE (108)..(108): “H” or “P”, predominantly H
  MISC_FEATURE (109)..(109): “E” or “R”, predominantly R
  MISC_FEATURE (110)..(110): “Q” or “A”, predominantly A
  MISC_FEATURE (111)..(111): “A” or “G”, predominantly A
  MISC_FEATURE (112)..(112): “E” or “G”, predominantly E
  MISC_FEATURE (113)..(113): “P” or “A”, predominantly A
  MISC_FEATURE (114)..(114): “D” or “R”, predominantly R
  MISC_FEATURE (115)..(115): “V” or “R”, predominantly R
  MISC_FEATURE (116)..(116): “S” or “E”, predominantly E
  MISC_FEATURE (117)..(117): “I” or “H”, predominantly H
  MISC_FEATURE (118)..(118): “S” or “I”, predominantly I
  MISC_FEATURE (119)..(119): “L” or “A”, predominantly A
  MISC_FEATURE (120)..(120): “S” or “L”, predominantly L
  MISC_FEATURE (121)..(121): “I” or “H”, predominantly H
  MISC_FEATURE (122)..(122): “A” or “R”, predominantly A
  MISC_FEATURE (124)..(124): “P” or “A”, predominantly A
  MISC_FEATURE (125)..(125): “C” or “V”, predominantly C
  MISC_FEATURE (126)..(126): “G” or “R”, predominantly R
  MISC_FEATURE (127)..(127): “S” or “Q”, predominantly Q
  MISC_FEATURE (128)..(128): “A” or “R”, predominantly A
  MISC_FEATURE (130)..(130): “F” or “L”, predominantly L
  MISC_FEATURE (131)..(131): “A” or “R”, predominantly A
  MISC_FEATURE (132)..(132): “T” or “H”, predominantly H
  MISC_FEATURE (133)..(133): “A” or “R”, predominantly A
  MISC_FEATURE (135)..(135): “A” or “G”, predominantly A
  MISC_FEATURE (136)..(136): “A” or “G”, predominantly A
  MISC_FEATURE (137)..(137): “P” or “A”, predominantly A
  MISC_FEATURE (138)..(138): “S” or “V”, predominantly S
  MISC_FEATURE (139)..(139): “T” or “D”, predominantly D
  MISC_FEATURE (140)..(140): “V” or “G”, predominantly G
  MISC_FEATURE (141)..(141): “T” or “D”, predominantly D
  MISC_FEATURE (142)..(142): “T” or “D”, predominantly D
  MISC_FEATURE (143)..(143): “T” or “D”, predominantly D
  MISC_FEATURE (144)..(144): “V” or “R”, predominantly R
  MISC_FEATURE (145)..(145): “A” or “G”, predominantly A
  MISC_FEATURE (146)..(146): “P” or “A”, predominantly A
  MISC_FEATURE (148)..(148): “G” or “R”, predominantly R
  MISC_FEATURE (149)..(149): “G” or “R”, predominantly R
  MISC_FEATURE (150)..(150): “Q” or “P”, predominantly Q
  MISC_FEATURE (151)..(151): “Q” or “A”, predominantly A
  MISC_FEATURE (152)..(152): “V” or “G”, predominantly G
  MISC_FEATURE (153)..(153): “P” or “A”, predominantly A
  MISC_FEATURE (154)..(154): “S” or “Q”, predominantly Q
  MISC_FEATURE (155)..(155): “Q” or “P”, predominantly Q
  MISC_FEATURE (156)..(156): “Y” or “V”, predominantly Y
  MISC_FEATURE (157)..(157): “W” or “L”, predominantly L
  MISC_FEATURE (158)..(158): “I” or “D”, predominantly D
  MISC_FEATURE (160)..(160): “S” or “L”, predominantly L
  MISC_FEATURE (161)..(161): “A” or “R”, predominantly A
  MISC_FEATURE (162)..(162): “A” or “R”, predominantly A
  MISC_FEATURE (163)..(163): “E” or “R”, predominantly R
  MISC_FEATURE (164)..(164): “I” or “D”, predominantly D
  MISC_FEATURE (165)..(165): “L” or “P”, predominantly L
  MISC_FEATURE (166)..(166): “V” or “R”, predominantly R
  MISC_FEATURE (167)..(167): “G” or “R”, predominantly R
  MISC_FEATURE (168)..(168): “S” or “L”, predominantly L
  MISC_FEATURE (169)..(169): “T” or “H”, predominantly H
  MISC_FEATURE (170)..(170): “Q” or “A”, predominantly A
  MISC_FEATURE (171)..(171): “F” or “V”, predominantly F
  MISC_FEATURE (172)..(172): “S” or “L”, predominantly L
  MISC_FEATURE (173)..(173): “C” or “L”, predominantly C
  MISC_FEATURE (174)..(174): “A” or “R”, predominantly A
  MISC_FEATURE (175)..(175): “V” or “R”, predominantly R
  MISC_FEATURE (176)..(176): “C” or “L”, predominantly C
  MISC_FEATURE (177)..(177): “N” or “Q”
  MISC_FEATURE (178)..(178): “K” or “Q”, predominantly Q
  MISC_FEATURE (179)..(179): “T” or “D”, predominantly D
  MISC_FEATURE (180)..(180): “F” or “L”, predominantly L
  MISC_FEATURE (181)..(181): “N” or “Q”
  MISC_FEATURE (182)..(182): “R” or “P”, predominantly R
  MISC_FEATURE (183)..(183): “F” or “L”, predominantly L
  MISC_FEATURE (184)..(184): “N” or “Q”
  MISC_FEATURE (185)..(185): “N” or “Q”
  MISC_FEATURE (186)..(186): “M” or “H”, predominantly H
  MISC_FEATURE (187)..(187): “Q” or “A”, predominantly A
  MISC_FEATURE (188)..(188): “M” or “D”, predominantly D
  MISC_FEATURE (189)..(189): “H” or “A”, predominantly A
  MISC_FEATURE (190)..(190): “M” or “H”, predominantly H
  MISC_FEATURE (191)..(191): “W” or “V”, predominantly W
  MISC_FEATURE (193)..(193): “H” or “P”, predominantly H
  MISC_FEATURE (194)..(194): “G” or “R”, predominantly R
  MISC_FEATURE (195)..(195): “S” or “L”, predominantly L
  MISC_FEATURE (196)..(196): “Q” or “A”, predominantly A
  MISC_FEATURE (197)..(197): “Y” or “V”, predominantly Y
  MISC_FEATURE (198)..(198): “R” or “P”, predominantly R
  MISC_FEATURE (199)..(199): “K” or “Q”, predominantly Q
  MISC_FEATURE (201)..(201): “S” or “L”, predominantly L
  MISC_FEATURE (202)..(202): “E” or “R”, predominantly R
  MISC_FEATURE (203)..(203): “S” or “V”, predominantly S
  MISC_FEATURE (204)..(204): “L” or “P”, predominantly L
  MISC_FEATURE (205)..(205): “R” or “A”, predominantly A
  MISC_FEATURE (206)..(206): “G” or “R”, predominantly R
  MISC_FEATURE (207)..(207): “A” or “R”, predominantly A
  MISC_FEATURE (208)..(208): “I” or “D”, predominantly D
  MISC_FEATURE (209)..(209): “T” or “H”, predominantly H
  MISC_FEATURE (210)..(210): “V” or “G”, predominantly G
  MISC_FEATURE (212)..(212): “T” or “H”, predominantly H
  MISC_FEATURE (213)..(213): “A” or “G”, predominantly A
  MISC_FEATURE (214)..(214): “P” or “A”, predominantly A
  MISC_FEATURE (215)..(215): “P” or “A”, predominantly A
  MISC_FEATURE (216)..(216): “A” or “G”, predominantly A
  MISC_FEATURE (217)..(217): “S” or “V”, predominantly S
  MISC_FEATURE (218)..(218): “L” or “A”, predominantly A
  MISC_FEATURE (219)..(219): “M” or “D”, predominantly D
  MISC_FEATURE (220)..(220): “R” or “A”, predominantly A
  MISC_FEATURE (221)..(221): “L” or “A”, predominantly A
  MISC_FEATURE (222)..(222): “P” or “A”, predominantly A
  MISC_FEATURE (223)..(223): “C” or “V”, predominantly C
  MISC_FEATURE (224)..(224): “Y” or “L”, predominantly L
  MISC_FEATURE (225)..(225): “C” or “L”, predominantly C
  MISC_FEATURE (226)..(226): “C” or “L”, predominantly C
  MISC_FEATURE (227)..(227): “A” or “R”, predominantly A
  MISC_FEATURE (228)..(228): “E” or “R”, predominantly R
  MISC_FEATURE (230)..(230): “C” or “V”, predominantly C
  MISC_FEATURE (231)..(231): “R” or “P”, predominantly R
  MISC_FEATURE (232)..(232): “N” or “E”
  MISC_FEATURE (233)..(233): “N” or “Q”
  MISC_FEATURE (234)..(234): “I” or “H”, predominantly H
  MISC_FEATURE (235)..(235): “E” or “R”, predominantly R
  MISC_FEATURE (236)..(236): “H” or “A”, predominantly A
  MISC_FEATURE (238)..(238): “R” or “A”, predominantly A
  MISC_FEATURE (239)..(239): “A” or “C”, predominantly A
  MISC_FEATURE (240)..(240): “R” or “S”, predominantly R
  MISC_FEATURE (242)..(242): “L” or “P”, predominantly L
  MISC_FEATURE (243)..(243): “K” or “Q”, predominantly Q
  MISC_FEATURE (244)..(244): “D” or “G”, predominantly D
  MISC_FEATURE (245)..(245): “F” or “L”, predominantly L
  MISC_FEATURE (246)..(246): “R” or “P”, predominantly R
  MISC_FEATURE (247)..(247): “T” or “D”, predominantly D
  MISC_FEATURE (248)..(248): “L” or “A”, predominantly A
  MISC_FEATURE (249)..(249): “Q” or “A”, predominantly A
  MISC_FEATURE (250)..(250): “T” or “D”, predominantly D
  MISC_FEATURE (251)..(251): “H” or “A”, predominantly A
  MISC_FEATURE (252)..(252): “Y” or “L”, predominantly L
  MISC_FEATURE (253)..(253): “R” or “P”, predominantly R
  MISC_FEATURE (254)..(254): “R” or “P”, predominantly R
  MISC_FEATURE (255)..(255): “K” or “E”, predominantly E
  MISC_FEATURE (256)..(256): “G” or “A”, predominantly A
  MISC_FEATURE (257)..(257): “A” or “R”, predominantly A
  MISC_FEATURE (259)..(259): “P” or “A”, predominantly A
  MISC_FEATURE (260)..(260): “Y” or “A”, predominantly A
  MISC_FEATURE (261)..(261): “A” or “V”, predominantly A
  MISC_FEATURE (262)..(262): “C” or “R”, predominantly R
  MISC_FEATURE (263)..(263): “R” or “V”, predominantly R
  MISC_FEATURE (264)..(264): “R” or “P”, predominantly R
  MISC_FEATURE (265)..(265): “C” or “A”, predominantly A
  MISC_FEATURE (266)..(266): “G” or “V”, predominantly G
  MISC_FEATURE (267)..(267): “K” or “R”, predominantly R
  MISC_FEATURE (268)..(268): “R” or “E”, predominantly R
  MISC_FEATURE (269)..(269): “F” or “A”, predominantly A
  MISC_FEATURE (270)..(270): “A” or “V”, predominantly A
  MISC_FEATURE (271)..(271): “V” or “R”, predominantly R
  MISC_FEATURE (273)..(273): “G” or “A”, predominantly A
  MISC_FEATURE (274)..(274): “D” or “R”, predominantly R
  MISC_FEATURE (275)..(275): “W” or “R”, predominantly R
  MISC_FEATURE (276)..(276): “R” or “L”, predominantly R
  MISC_FEATURE (277)..(277): “T” or “A”, predominantly A
  MISC_FEATURE (279)..(279): “E” or “P”, predominantly E
  MISC_FEATURE (280)..(280): “K” or “R”, predominantly R
  MISC_FEATURE (281)..(281): “N” or “E”
  MISC_FEATURE (282)..(282): “C” or “E”, predominantly C
  MISC_FEATURE (283)..(283): “G” or “L”, predominantly G
  MISC_FEATURE (285)..(285): “L” or “Q”, predominantly Q
  MISC_FEATURE (286)..(286): “W” or “A”, predominantly A
  MISC_FEATURE (287)..(287): “F” or “L”, predominantly L
  MISC_FEATURE (288)..(288): “C” or “V”, predominantly C
  MISC_FEATURE (289)..(289): “V” or “L”, predominantly L
  MISC_FEATURE (290)..(290): “C” or “R”, predominantly R
  MISC_FEATURE (291)..(291): “G” or “L”, predominantly G
  MISC_FEATURE (292)..(292): “S” or “R”, predominantly R
  MISC_FEATURE (293)..(293): “D” or “F”, predominantly D
  MISC_FEATURE (294)..(294): “F” or “R”, predominantly R
  MISC_FEATURE (295)..(295): “K” or “L”, predominantly L
  MISC_FEATURE (296)..(296): “H” or “Q”, predominantly Q
  MISC_FEATURE (297)..(297): “K” or “A”, predominantly A
  MISC_FEATURE (298)..(298): “R” or “Q”, predominantly R
  MISC_FEATURE (299)..(299): “S” or “A”, predominantly A
  MISC_FEATURE (301)..(301): “K” or “A”, predominantly A
  MISC_FEATURE (302)..(302): “D” or “Q”
  MISC_FEATURE (303)..(303): “H” or “G”, predominantly G
  MISC_FEATURE (304)..(304): “V” or “P”, predominantly P
  MISC_FEATURE (306)..(306): “S” or “P”, predominantly P
  MISC_FEATURE (307)..(307): “F” or “L”, predominantly L
  MISC_FEATURE (308)..(308): “G” or “L”, predominantly G
  MISC_FEATURE (309)..(309): “G” or “R”, predominantly R
  MISC_FEATURE (310)..(310): “G” or “R”, predominantly R
  MISC_FEATURE (311)..(311): “H” or “G”, predominantly G
  MISC_FEATURE (313)..(313): “P” or “C”, predominantly C
  MISC_FEATURE (314)..(314): “H” or “A”, predominantly A
  MISC_FEATURE (315)..(315): “I” or “A”, predominantly A
  MISC_FEATURE (316)..(316): “V” or “H”, predominantly H
  MISC_FEATURE (317)..(317): “E” or “R”, predominantly R
  MISC_FEATURE (318)..(318): “S” or “R”, predominantly R
  MISC_FEATURE (320)..(320): “A” or “R”, predominantly A
  MISC_FEATURE (321)..(321): “G” or “R”, predominantly R
  MISC_FEATURE (322)..(322): “V” or “R”, predominantly R
  MISC_FEATURE (323)..(323): “G” or “R”, predominantly R
  MISC_FEATURE (324)..(324): “G” or “R”, predominantly R
  MISC_FEATURE (325)..(325): “A” or “R”, predominantly A
  MISC_FEATURE (326)..(326): “D” or “R”, predominantly R
  MISC_FEATURE (327)..(327): “D” or “R”, predominantly R
  MISC_FEATURE (328)..(328): “D” or “R”, predominantly R
  MISC_FEATURE (329)..(329): “E” or “R”, predominantly R
  MISC_FEATURE (330)..(330): “E” or “R”, predominantly R
  MISC_FEATURE (331)..(331): “D” or “G”, predominantly D
  MISC_FEATURE (332)..(332): “D” or “G”, predominantly D
  MISC_FEATURE (333)..(333): “D” or “R”, predominantly R
  MISC_FEATURE (334)..(334): “E” or “R”, predominantly R
  MISC_FEATURE (335)..(335): “H” or “R”, predominantly R
  MISC_FEATURE (336)..(336): “D” or “T”, predominantly D
  MISC_FEATURE (337)..(337): “D” or “R”, predominantly R
  MISC_FEATURE (338)..(338): “A” or “R”, predominantly A
  MISC_FEATURE (339)..(339): “D” or “R”, predominantly R
  MISC_FEATURE (340)..(340): “L” or “R”, predominantly R
  MISC_FEATURE (341)..(341): “Q” or “P”, predominantly Q
  MISC_FEATURE (342)..(342): “M” or “A”, predominantly A
  MISC_FEATURE (343)..(343): “V” or “D”, predominantly D
  MISC_FEATURE (344)..(344): “D” or “G”, predominantly D
  MISC_FEATURE (345)..(345): “D” or “G”, predominantly D
  MISC_FEATURE (346)..(346): “N” or “R”, predominantly R
  MISC_FEATURE (347)..(347): “G” or “Q”, predominantly Q
  MISC_FEATURE (348)..(348): “G” or “R”, predominantly R
  MISC_FEATURE (349)..(349): “S” or “R”, predominantly R
  MISC_FEATURE (350)..(350): “D” or “L”, predominantly D
  MISC_FEATURE (351)..(351): “M” or “R”, predominantly R
  MISC_FEATURE (352)..(352): “V” or “H”, predominantly H
  MISC_FEATURE (353)..(353): “V” or “G”, predominantly G
  MISC_FEATURE (354)..(354): “V” or “R”, predominantly R
MQHSYRAMHPYTHPYQHHAASAVPPSNKQHGDDDDDQSMLLLSLWPPGHHHHSXXXXXXXPXXXXXXXXXXXXXXXXXXXXPXXXXXXXXXXXXXXXXXXXXXXXXXXXXXXXXXXXXXXXXPXXXXXGXXXXPXXXXXXXXXXXXGXXXXXXXXXXXPXXXXXXXXXXXXXXXXXXXXXXXXXXXXXXXXGXXXXXXXGXXXXXXXXXXGXXXXXXXXXXXXXXXXXGXXXXXXXPXXXPXXXXXXXXXXXXXXXXRXXXXXXXXXXXXXRXXXXXHXXXXXRXXXXXXXXXXXXXXXLXXXXRXXXXXXAXXXXXXVXXXXXXXXXXXXXXXXXXXXXXXXXXXXXXXXXXX

SEQ ID NO: 31 - DNA, 20 bp, Artificial Sequence
  Forward primer
caaccaagctctgatagagt

SEQ ID NO: 32 - DNA, 19 bp, Artificial Sequence
  Reverse primer
gaagaatctcgtgctttca

SEQ ID NO: 33 - DNA, 2960 bp, Oryza sativa Japonica
ggagtgtacattaaatttgggttggcatatttgtagattgatattgatctttttaacaatttttatatccttttaaaaatgaggggatgtacccctggaaagacattttcacatatgtaataaatactcacttcgtcgcacataaaaaaaactaacttctattctaattaagaaactaacttttctgtagctagtagttgtttttttaagttcatatacatatacatatacatactcctacatgtatgatgtagagagaggagaggtcaatactggagaaatgatttttgctcggtggtccgaaaaccacaataatccatcctgattcgaataagaaccagacaaaagatcatttgttgtcccggttaaacgagtacgtgtacttcttcccttctataaataccaaatctaatacaaaatatgatacatttttctgttcatatttataatactatcatattttatgagatgaaggaaataggtcatcgggagtcgtgctggcaagggtggggcccagctgagggacaagaccgggggaggagagggggacaggcaggtgggtcccgacccggtgccattgccattgccatgcgaagggacgcgtagctaggtgtgtggtcccatgcatgtatgcgtggcaaacgttgcgttgtcccctccctgccctaatgcctccaacgcctacctcctgcgatgctagctgcctctgctccccccccccccccccctctatcgcattcgcatcgcatgctacagtacaggcctcatctccctttcgcagcagcagcagcgtccatccatccgatccatctatcgaccatcacactctcgtccatccatgtcctatgtactccccgtagtacatataaccaccggactctccctctctcatcttgtcgtgtcaattaagcttcttgcattggtcaatgtatacgtatgcatatatagtacttcggttttataatgtaataatctagtatccaatgagacatatccctagtacggagaatctgcctagatttgtaatactagctagaatgtgtgcaattcggtgtactagattactactacctccttgcacaaatgtatgacgttgattaaccaacgtcaaacaaaaaaaaatatggacggagtactccctccgtcccataaaaaatcaacctagtatcggatgtgacacatcctaatttacgaatatggacatatatatatatatatatatatatatatatatatatatatatatatatatatatatatatatatacacacacacagattcatcatactagattatgtcacatccagttctagattcgtattttatagaacagagggagtatattttaagacgaaactagtatatatatagctgtatgtactacctttgtgttcttagcacacatactagattcgttcccgaatataagcattcctacaattcaaattctatatgcaatgtaagtattttttttacattggttccattgcacatgaataggggaatccaagaaattaaaaaggttgaaaatttaatcaatgaggtgaatgaaagagaatatttccctttatccttaatcattcttaaacaacctaaaaatcatctattttggactatcttttgacaattggttcttccccattcaccatgaacttatatatcttcataaaatgcagatatgtatggttttacaaaatttggcatctaaatcatctattttgcgttaacttgacaccaaattaacaagagttaccatgtaagttaaatatatattagatgtacaatgtaaaacaaaaaataataccattattatgttgccctaaaattggggggtatttgcatgaactggttttaaaagttcaaggtgagtacataaaatgtatggtttgtagccaacggttgatttatagactcaaagaagggtaaaatagactttggctctattggggggagcttctagctattgcagctttctctagtagaagctccaattcttccaaacaatttatattgtcagaaaatgaactacagtcaaaaactgggttttataacttctctagattcttagaagccgactagcaaaccgcagcttcttaaaatttttagctccccaaacaagacctttaaatgcacaaaaaaaaggtactcctccgtcctaaaatataagcattcttgaacttcgacacagtcatcgagatactactttaaccaacaatatctataaaaataaaatattttaaataaaaagagttacatatacgatagtttgattaatgataattctaataacatcaattttacatgattgaattttttaattttttttactattaatagtcaaagttaaaaatgattgactcggtactatgctaaaaatgcttatattttggtacggagggagtagaacaatgcgcactaaataacacatattcttgtaacttgcattaacccaccacatgattgttagggtatgtacgtagctacataaaaatgtcattgcacgtgcaagctccccctacactacactacactacactacatacccccaccctaccaatcattcaactatacctatataattttctcactcctctctctagctatatataccaccctctaccatgtccatggacacccaacattcacagacattcataacatactacatatcacctagagtagattacatatcacctagagtagatcaagcaaggcccctccatctcccctcttccaacacttgttttgctcccccccccccctctctctctctctctctctctctcttccattcaaccttccaaactctctcactcactcatcttagagagagaaagaagagagagagagtgaagagaagtgaagctatctagctagctagagagagagaattgtagagtgagagtgagggaattaattatcaagaagaatgcatagcagcagcaggtacagagcaatg

SEQ ID NO: 34 - DNA, 729 bp, Oryza sativa Japonica
gcttcttaattattttgtgtttaattaattactactttggatcgagttagtttgttgttgttgttgtggaggaggaagatgaccgtacagtctagctaggtataggctactagtagtagtagactactcctagatggctaggctaggcagccatgggtgtacggccagggagtagcagagctagggttagagagagagagagaatgagagagagggagacagatgtgtacggtttggtgtcatggtcatttcttgtacctgtcagtttggtctttatctgcgtccaaagttctaccactgtttcattgtgtctcgtgctctctctctctctctctctctctctctctagccacgcttgcttcctgcttctccttcaattcccttgtcaaagacacatccaagatcagcgaaaaggctacaccttaattagagagagaatcatacacatacttcctccgttttgtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgct

(19) United States
(12) Patent Application Publication
(10) Pub. No.: US 2023/0012791 A1
(43) Pub. Date: Jan. 19, 2023

(54) Modified Plants

(71) Applicant: Oxford University Innovation Limited, Botley, Oxfordshire (GB)

(72) Inventors: Jane Alison LANGDALE, Oxford, Oxfordshire (GB); Daniela Loredana VLAD, Botley, Oxfordshire (GB)

(21) Appl. No.: 17/629,593

(22) PCT Filed: Jul. 24, 2020
(86) PCT No.: PCT/IB2020/056992

§ 371 Date: Jan. 24, 2022

(30) Foreign Application Priority Data
Jul. 26, 2019 (AU) 2019902678
Oct. 15, 2019 (AU) 2019903883

(51) Int. Cl. C12N 15/82

(52) U.S. Cl. CPC C12N 15/8213; C12N 15/8218; C12N 15/8269

(57) ABSTRACT

The present invention provides modified (e.g. genetically modified) plant cells and plants, the plants being characterised by an increase in any one or more of: bundle sheath cell numbers, bundle sheath volume, bundle sheath area, vein density, and/or the proportion of lateral veins, as compared to wild-type counterparts.

### TECHNICAL FIELD

The present invention relates to the field of biotechnology and the engineering of desirable traits into plants. More specifically, the present invention relates to plant cells and plants having modified gene expression and/or modified protein activity, along with methods for their production. Without limitation, modified plants according to the present invention may be characterised by an increase in any one or more of: bundle sheath cell numbers, bundle sheath volume, bundle sheath area, vein density, and/or the proportion of lateral veins, as compared to wild-type counterparts.

### BACKGROUND

Underpinning virtually all life on earth is the process of photosynthesis, whereby plants, algae and photosynthetic bacteria use light energy to fix inorganic carbon dioxide (CO₂) into organic sugars. The most common form of photosynthesis is termed C₃ because CO₂ is fixed by the enzyme ribulose-1,5-bisphosphate carboxylase/oxygenase (RuBisCO) into a three carbon compound. Despite its ubiquity, however, C₃ photosynthesis is hampered by the inability of RuBisCO to faithfully distinguish between CO₂ and O₂, leading to competing carboxylation and oxygenation reactions. Oxygenation, which is elevated in hot, dry environments, yields phosphoglycolate which has to be recycled in an energy-consuming process termed photorespiration. Both temperature increases and drought stress thus increase photorespiration and decrease photosynthetic efficiency in C₃ plants.

A variety of strategies to concentrate CO₂ around RuBisCO and thus to reduce photorespiration can be found in nature. These include the carbon-concentrating mechanisms of many cyanobacteria and green algae, and both crassulacean acid metabolism (CAM) and C₄ photosynthesis in flowering plants. Of these divergent strategies, C₄ photosynthesis is the most agronomically important, because productive crop species such as maize, sorghum and sugarcane am C₄, as are many weeds. The most common form of the C₄ pathway, involves the spatial separation of metabolic reactions between two cell-types organized within a characteristic leaf anatomy known as Kranz. CO₂ is initially fixed by an O₂-insensitive carboxylase into a four carbon compound (hence C₄ pathway) in the outer mesophyll cells of the leaf, and then the C₄ compound is transferred to inner vascular sheath cells, where it is decarboxylated to release CO₂ for re-fixation by RuBisCO. This intercellular C₄ shuttle concentrates CO₂ at the site of RuBisCO, leading to low levels of photorespiration and enhanced photosynthetic efficiency relative to C₃ plants, particularly in hot and/or dry environments. The more efficient C₄ pathway, which evolved from the C₃ pathway, accounts for ˜25% of primary terrestrial productivity on the planet despite being used by only 3% of species.

In C₃ leaves of monocots and eudicots, veins are generally encircled by layer(s) of vascular sheath cells (mestome sheath and/or bundle sheath) and in the majority of cases are separated from adjacent veins by at least six mesophyll cells. RuBisCO and hence C₃ carbon fixation primarily occur in the mesophyll cells whereas vascular sheath cells load metabolites into the vein, provide structural support and at least in some species are proposed to facilitate cavitation repair during drought stress. Although the C₄ photosynthetic pathway can also operate in the context of single cells, with initial fixation and subsequent decarboxylation reactions split between distinct intracellular compartments, the majority of C₄ species compartmentalize reactions between two distinct cell-types in the leaf. In C₄ leaves with Kranz anatomy, concentric rings of photosynthetic vascular sheath and mesophyll cells surround each vein and adjacent veins are often separated by just two mesophyll cells. C₄ Kranz anatomy thus differs from C₃ anatomy with respect to vein spacing patterns across the leaf, cell-type specification around veins, and cell-specific organelle function.

The physical features of Kranz anatomy and mechanisms of C₄ photosynthesis have been relatively well characterised. Nonetheless, while genes encoding enzymes of the C₄ pathway were identified over 30 years ago forward genetic screens over many years have failed to provide significant insight into the genes that regulate Kranz anatomy.

A need exists to identify gene/s capable of regulating Kranz anatomy. The manipulation of these gene/s may provide a means of enhancing Kranz-like anatomy in the leaves of plants.

### SUMMARY OF THE INVENTION

The present inventors have demonstrated that loss of function of certain transcription factors in plants provides a means of increasing any one or more of: bundle sheath cell numbers, bundle sheath volume, bundle sheath area, vein density, and/or the proportion of lateral veins. This in turn may lead to increased photosynthetic capacity in some environments.

Accordingly, the invention as described herein relates at least in part to the following embodiments 1-43 numbered/listed below:

Embodiment 1: A plant cell modified to inhibit or prevent expression of a gene as defined in SEQ ID NO: 1 or an orthologue thereof, and/or activity of a protein encoded by the gene, within the cell.

Embodiment 2: The plant cell of embodiment 1, wherein the plant cell is genetically modified to inhibit or prevent said expression of the gene or orthologue thereof.

Embodiment 3: The plant cell of embodiment 1 or embodiment 2, wherein the plant cell is genetically modified by knockout of said expression of the gene or orthologue thereof.

Embodiment 4: The plant cell of embodiment 3, wherein the knockout of said expression arises from deletion of all or a part of the gene.

Embodiment 5: The plant cell of embodiment 3 or embodiment 4, wherein the knockout of said expression arises from a frameshift mutation, a stop mutation, or a combination thereof, introduced into the gene.

Embodiment 6: The plant cell of any one of embodiments 1 to 5, wherein the plant cell is engineered to express inhibitory RNA molecules capable of knocking down expression of the gene in the plant cell.

Embodiment 7: The plant cell of embodiment 6, wherein the inhibitory RNA molecules comprise microRNA or small interfering RNA (siRNA).

Embodiment 8: The plant cell of embodiment 6 or embodiment 7, wherein the plant cell comprises an expression vector for expressing the inhibitory RNA molecules.

Embodiment 9: The plant cell of any one of embodiments 1 to 8, wherein the plant cell is engineered to express an antagonist capable of inhibiting said activity of the protein encoded by the gene.

Embodiment 10: The plant cell of embodiment 9, wherein the plant cell comprises an expression vector for expressing the antagonist.

Embodiment 11: The plant cell of any one of embodiments 1 to 10, wherein the gene comprises or consists of a nucleotide sequence having at least: 70%, 75%, 80%, 85%, 87%, 89%, 90%, 91%, 92%, 93%, 94%, 95%, 96%, 97%, 98%, 99%; sequence identity to a nucleotide sequence as defined in SEQ ID NO: 1.

Embodiment 12: The plant cell of any one of embodiments 1 to 11, wherein the protein comprises or consists of an amino acid sequence:

(i) as defined in SEQ ID NO: 3; or
(iii) having at least: 70%, 75%, 80%, 85%, 87%, 89%, 90%, 91%, 92%, 93%, 94%, 95%, 96%, 97%, 98%, 99%; sequence identity to an amino acid sequence as defined in SEQ ID NO: 3.

Embodiment 13: The plant cell of any one of embodiments 1 to 12, wherein the gene and/or protein comprises one or more ethylene-responsive element binding factor-associated amphiphilic repression (EAR) domains, optionally wherein the one or more EAR domains comprise a motif sequence defined by LxLxL (e.g. LLLSL), DLNxxP, or an overlap of LxLxL and DLNxxP.

Embodiment 14: The plant cell of any one of embodiments 1 to 13, wherein the expression of the gene and/or the activity of the protein is inhibited or prevented by deletion of all or a portion of one or more of said EAR domains from the gene and/or protein, or, binding of antagonist/s to the EAR domain/s.

Embodiment 15: The plant cell of embodiment 13 or embodiment 14, wherein the plant cell is a: Poaceae/Gramineae (grass), Linaceae, Euphorbiaceae, or Bromeliaceae; family member.

Embodiment 16: The plant cell of any one of embodiments 13 to 15, wherein the plant cell is any of a: rice, wheat, maize, barley, oat, rye, common millet, finger millet, teff, sugar cane, or sorghum; cell.

Embodiment 17: The plant cell of any one of embodiments 13 to 16, wherein the plant cell is any of: Ananas comosus, Manihot esculenta, Setaria italica, Oryza sativa, Oryza sativa japonica, Oryza sativa Kitaake, Brachypodium distchyon, Zea mays, Sorghum bicolor, Anasus comosus, Linum usitatissimum.

Embodiment 18: The plant cell of any one of embodiments 13 to 17, wherein the plant cell is genetically modified to express auxin or an analogue thereof (e.g. 4-dichlorophenoxyacetic acid (2,4-D), 1-naphthaleneacetic acid (1-NAA), indole-3-acetic acid [IAA], 2,4-D, 1-NAA), optionally comprising an expression vector encoding said auxin or analogue thereof.

Embodiment 19: The plant cell of any one of embodiments 1 to 18, wherein the plant cell is a monocotyledonous plant cell.

Embodiment 20: A method for providing the plant cell of any one of embodiments 1 to 19, the method comprising:

obtaining vectors comprising one or more sequences capable of inhibiting or preventing expression of a gene as defined in SEQ ID NO: 1 or an orthologue thereof, and/or activity of a protein encoded by the gene, within the plant cell;

subjecting a population of plant cells to a transformation procedure using the vectors to thereby provide a subpopulation of plant cells transformed by the vectors; and

selecting a member of said subpopulation of plant cells from said population based on expression of a selectable marker indicative of said transformation, to thereby provide the plant cell.

Embodiment 21: The method of embodiment 20, wherein the one or more sequences encode clustered regularly interspaced short palindromic repeats (CRISPR) guide RNA and a Cas9 nuclease, wherein the guide RNA is specific for the gene and comprises a Protospacer Adjacent Motif (PAM) specific for the Cas9 nuclease.

Embodiment 22: The method of embodiment 20, wherein the one or more sequences comprise a sequence capable of homologous recombination with the gene of the plant cell to thereby remove all or a portion the gene and thereby render the gene non-functional.

Embodiment 23: The method of embodiment 20, wherein the one or more sequences encode an antagonist of expression or activity of the protein.

Embodiment 24: The method of embodiment 23, wherein the antagonist of the protein expression is an inhibitory RNA molecule.

Embodiment 25: The method of embodiment 23, wherein the antagonist of the protein activity is a protein capable of binding to the protein to thereby inhibit or prevent said protein activity.

Embodiment 26: The method of any one of embodiments 20 to 25, wherein the gene and/or protein comprises one or more EAR domains, and said inhibiting or preventing comprises: removal of all or a part of each said EAR domain from the gene and/or protein, or, binding of antagonist/s to each said EAR domain.

Embodiment 27: The method of embodiment 26, wherein the plant cell is:

(i) a Poaceae/Gramineae (grass), Linaceae, Euphorbiaceae, or Bromeliaceae; family member;
(ii) any of a: rice, wheat, maize, barley, oat, rye, common millet, finger millet, teff, sugar cane, or sorghum; cell;
(iii) any of: Ananas comosus, Manihot esculenta, Setaria italica, Oryza sativa, Oryza sativa japonica, Oryza sativa Kitaake, Brachypodium distchyon, Zea mays, Sorghum bicolor, Anasus comosus, Linum usitatissimum.

Embodiment 28: The method of any one of embodiments 20 to 27, further comprising genetically engineering the plant cell to express auxin or an analogue thereof (e.g. 4-dichlorophenoxyacetic acid (2,4-D), 1-naphthaleneacetic acid (1-NAA), indole-3-acetic acid [IAA], 2,4-D, 1-NAA).

Embodiment 29: The method of embodiment 28, comprising transforming the plant cell with an expression vector encoding said auxin or analogue thereof.

Embodiment 30: The method of any one of embodiments 20 to 29, wherein the plant cell is any of: a leaf ground meristem cell, a leaf procambial initial cell, a vascular sheath cell, a bundle sheath cell, a mestome sheath cell, a mesophyll cell.

Embodiment 31: The method of any one of embodiments 20 to 30, wherein the plant cell is C₃ photosynthetic plant cell.

Embodiment 32: The method of any one of embodiments 20 to 30, wherein the plant cell is C₄ photosynthetic plant cell.

Embodiment 33: The plant cell of any one of embodiments 1 to 19, or the method of any one of embodiments 20 to 32, wherein the plant cell is a genus Oryza plant cell.

Embodiment 34: The plant cell of any one of embodiments 1 to 19 or 33, or the method of any one of embodiments 20 to 33, wherein the plant cell is a Oryza sativa plant cell.

Embodiment 35: The plant cell of any one of embodiments 1 to 19, 33, or 34, or the method of any one of embodiments 20 to 34, wherein the plant cell is a: Soy (Glycine max), Cotton (Gossypium hirsutum), Oilseed rape/Cannola (B. napus subsp. napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), Cannabis (Cannabis sativa), sugar beet (Beta vulgaris), oat (Avena sativa), rye (Secale cereale), peanut (Arachis hypogaea), Sunflower (Helianthus annuus), flax (Linum spp.), bean (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), Oryza sativa L ssp. indica, Oryza sativa L ssp. japonica, Oryza sativa Kitaake, Oryza sativa japonica, rubber (Hevea brasiliensis), or monocotyledonous; plant cell.

Embodiment 36: A genetically modified plant or a seed thereof comprising the plant cell of any one of embodiments 1 to 19 or 33 to 35.

Embodiment 37: Use of the plant cell of any one of embodiments 1 to 19 or 33 to 35 to generate a genetically modified plant.

Embodiment 38: A method for producing a genetically modified plant comprising obtaining the plant cell of any one of embodiments 1 to 19 or 33 to 35 and generating the genetically modified plant from said plant cell.

Embodiment 39: The use of embodiment 37 or the method of embodiment 38, wherein the genetically modified plant is C₃ photosynthetic plant.

Embodiment 40: The use of embodiment 37 or the method of embodiment 38, wherein the genetically modified plant is C₃ photosynthetic plant.

Embodiment 41: The use of embodiment 37 or 39, or the method of embodiment 38 or 39, wherein the genetically modified plant is a genus Oryza plant.

Embodiment 42: The use of any one of embodiments 37, 39 or 41, or the method of any one of embodiments 38, 39, or 41, wherein the genetically modified plant is a Oryza sativa or Oryza glaberrima plant.

Embodiment 43: The use of any one of embodiments 37 or 39 to 42, or the method of any one of embodiments 38 to 42, wherein the genetically modified plant is a: Soy (Glycine max), Cotton (Gossypium hirsutum), Oilseed rape/Cannola (B. napus subsp. napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), Cannabis (Cannabis sativa), sugar beet (Beta vulgaris), oat (Avena sativa), rye (Secale cereale), peanut (Arachis hypogaea), Sunflower (Helianthus annuus), flax (Linum spp.), bean (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), Oryza sativa L ssp. indica, Oryza sativa L ssp. japonica, Oryza sativa Kitaake, Oryza sativa japonica, rubber (Hevea brasiliensis), or monocotyledonous; plant.

### Definitions

As used in this application, the singular form “a”, “an” and “the” include plural references unless the context clearly dictates otherwise. For example, the term “cell” also includes multiple cells unless otherwise stated.

As used herein, the term “comprising” means “including”. Variations of the word “comprising”, such as “comprise” and “comprises” have correspondingly varied meanings. Thus, for example, a polynucleotide “comprising” nucleotide sequence ‘A’ may consist exclusively of nucleotide sequence ‘A’, or may include one or more additional nucleotide sequence/s, for example, nucleotide sequence ‘B’ and/or nucleotide sequence ‘C’.

As used herein, a “C₃ photosynthetic plant”, will be understood to encompass any plant in which all or the majority of photosynthesis is limited to C₃ photosynthesis. “C₃ photosynthesis” means a photosynthetic pathway which uses only the Calvin-Benson cycle for fixing carbon dioxide from air, providing a three-carbon compound. Cell types referred to herein as “C₃” will be understood to be from a “C₃ photosynthetic plant”.

As used herein, a “C₄ photosynthetic plant” will be understood to encompass any plant in which all or the majority of photosynthesis is limited to C₄ photosynthesis. “C₄ photosynthesis” means a photosynthetic pathway in which an intermediate four-carbon compound is used to transfer CO₂ to the site of CO₂ fixation through the Calvin-Benson cycle. C₄ photosynthesis commences with light-dependent reactions in mesophyll cells and the preliminary fixation of carbon dioxide to malate. Carbon dioxide is released from malate, where it is fixed again by Rubisco and the Calvin-Benson cycle. Cell types referred to herein as “C₄” will be understood to be from a “C₄ photosynthetic plant”. C₄ photosynthesis can occur in a single cell or can be distributed across multiple cells in a plant leaf.

As used herein a “nucleic acid” refers to deoxyribonucleotides or ribonucleotides and polymers thereof in either single-, double-stranded or triplexed form. The term may encompass nucleic acids containing known analogues of natural nucleotides having similar binding properties as the reference nucleic acid. A particular nucleic acid sequence may also implicitly encompass conservatively modified variants thereof (e.g. degenerate codon substitutions) and complementary sequences. The terms “nucleic acid”, “nucleic acid sequence” or “polynucleotide” may also be used interchangeably with gene, cDNA, and mRNA encoded by a gene.

As used herein the term “orthologue” refers to a gene that is related to a reference gene by descent from a common ancestral gene. Genes that are “orthologues” will be understood to be corresponding genes in different lineages resulting from speciation.

As used herein, the term “OsDOT1 orthogroup” will be understood to encompass the OsDOT1 gene as described herein and orthologues thereof.

As used herein, the term “analogue” in the context of a peptide or protein means an artificial or natural substance that resembles the peptide or protein in function. For example, a protein or peptide analogue may bind a receptor and thereby bring about the same or similar result as if a natural protein/peptide had bound to the receptor. In an embodiment such analogues may also resemble the protein/peptide in structure. Analogues contemplated in an embodiment of the present invention include fully or partially peptidomimetic compounds as well as peptides or proteins resembling a subject peptide in activity but comprising addition, deletion, or substitution of one or more amino acids compared to the subject peptide or protein. The term “analogue” as used herein with reference to nucleotide sequences encompasses sequences comprising addition, deletion, or substitution (including conservative amino acid substitutions) of one or more bases relative to a subject nucleotide sequence, wherein the encoded polypeptide resembles the polypeptide encoded by the subject nucleic acid molecule in function.

As used herein, a percentage of “sequence identity” will be understood to arise from a comparison of two sequences in which they are aligned to give a maximum correlation between the sequences. This may include inserting “gaps” in either one or both sequences to enhance the degree of alignment. The percentage of sequence identity may then be determined over the length of each of the sequences being compared. For example, a nucleotide sequence (“subject sequence”) having at least 95% “sequence identity” with another nucleotide sequence (“query sequence”) is intended to mean that the subject sequence is identical to the query sequence except that the subject sequence may include up to five nucleotide alterations per 100 nucleotides of the query sequence. In other words, to obtain a nucleotide sequence of at least 95% sequence identity to a query sequence, up to 5% (i.e. 5 in 100) of the nucleotides in the subject sequence may be inserted or substituted with another nucleotide or deleted.

As used herein, a regulatory sequence “operably linked” to another sequence means that a functional relationship exists between the two sequences such that the regulatory sequence has the capacity to exert an influence on the expression and/or localisation and/or activity of the sequence to which it is linked. For example, a promoter operably linked to a coding sequence will be capable of modulating the transcription of the coding sequence. A targeting peptide operably linked to a polypeptide will be capable of directing the polypeptide to a specific location (e.g. an organelle or cytoplasmic membrane).

### BRIEF DESCRIPTION OF THE FIGURES

Preferred embodiments of the present invention will now be described by way of example only, with reference to the accompanying figures wherein:

FIG. 1 relates to experiments using CRISPR/Cas9-mediated gene editing of OsDOT1. (A) Diagram outlining the position (highlighted purple regions) and direction (purple arrows) of the three sgRNAs targeting the OsDOT1 gene. The target sequences represented contain the PAM motif marked in purple italic. (B) Protein encoded by OsDOT1 highlighting the predicted EAR motif (red) and the C2H2 zinc-finger domain (blue). (C) Summary of alleles isolated and fixed in T1 progeny, with allele m3 being fixed from multiple independent transformation events. (D) Cross-sections depicting venation patterns in flag leaf-1 of WT and T1 plants carrying the Osdot1-m3 or Osdot1-m4 alleles, with the former being derived from four independent transgenic events.

FIG. 2 depicts a Southern blot analysis of T0 CRISPR lines. Hybridization of HindIII digested genomic with a DIGOXYGENIN-labelled fragment of the HYG gene as probe. T0 plants resulting from the same transformation event are identified by event number (first number after underscore [symbol] _ [symbol] ). Small letters indicate the allelism in the specific line, with a marking lines containing alleles m3 and m4, b marking lines containing allele m3, and c marking lines containing alleles m1 and m2. The distinct banding patterns in lines marked with a or b demonstrate that the four Osdot1-m3 mutants shown in FIG. 1D arose from independent transformation events.

FIG. 3 depicts phenotypic changes associated with the OsDOT1 knock-out alleles in plants according to embodiments of the present invention. (A) Box plot showing no effect on leaf width is associated with the loss of endogenous DOT1 function in rice. (B) Effects of Osdot1-m1 and Osdot1-m2 mutant alleles on vein density when compared to wild type plants at similar developmental stage. (C) Scale bars, 100 μm. Flag leaf cross-section highlighting additional lateral veins developing in the Osdot1-m1 background (marked by red arrow). (D) Bar chart illustrating average proportions of each vein categories present in wild type versus mutant plants. Rank 2 intermediate veins are only found in Osdot1 mutants.

FIG. 4 is a graph showing the number of bundle sheath (BS) cells in contact with the mesophyll per mm leaf width in wild type (wt) Kitaake rice, and mutant Kitaake rice strains according to embodiments of the present invention (Osdot1-m1—Kitaake rice OsDOT1 gene mutant allele 1; Osdot1-m2—Kitaake rice OsDOT1 gene mutant allele 2).

FIG. 5 demonstrates that changes in plant height are not linked to the OsDOT1 knock-out alleles. (A, B) Illustrates reduced plant height associated with the Osdot1-m1 and Osdot1-m2 alleles when compared to wild type. (C) Plant growth is restored in homozygous Osdot1-m2 (F2) after backcross to wild type. (D) Bar chart showing average plant size measured as the height of the first mature inflorescence in segregating F2 wild types, heterozygous and mutant plants. Scale bars, 5 cm.

FIG. 6 provides images showing that the vein patterning phenotype evident in gene edited plants of the present invention is not associated with reduced plant height. (A) Shows reduced plant height associated with the Osdot1-m2 alleles when compared to wild type. (B) Plant growth in T1 homozygous lines isolated from a second CRISPR experiment (same lines as shown in FIG. 1D). Scale bars, 5 cm.

FIG. 7 shows Osdot1-m1 and Osdot1-m3 mutant plants according to embodiments of the invention.

FIG. 8 shows wild-type Oryza sativa japonica DOT1 gene (A), complementary DNA (cDNA) (B) and protein (C) sequences. Underlined sequence in (B)=EAR domain.

FIG. 9 shows wild-type Oryza sativa japonica DOT1 promoter (A) and 3′UTR fused to tNOS terminator (B) sequences used for complementation attempts. Golden Gate fusion sites are represented in bold italic. In (A) sequence domesticated sites are shown in bold lowercase and variation at microsatellites in underlined lowercase and in (B) the tNOS terminator sequence is shown in lowercase.

FIG. 10 is a Vista plot showing conserved regions at the DOT1 locus (LOC_Os09g13680 in rice). Analysis of corresponding genomic regions from Z. mays, S. viridis and P. virgatum pinpoint a highly conserved sequence in the 3′UTR (blue arrow). Sequence is misannotated as non-coding in the image above. The Vista Plot software illustrates conserved non-coding regions in pink, exons in dark blue and UTRs in light blue.

FIG. 11 compares the effect of exogenous auxin (0.1 μM naphthaleneacetic acid (NAA)) on the phenotype of wild type and Osdot1 mutant (Osdot1-m2) plants. Six seedlings on left—WT Kitaake; Six seedlings in middle—2 homozygous Osdot1-m2 lines—3 seedlings of each; Six seedlings on right—2 heterozygous Osdot1-m2 lines—3 seedlings of each. Genotypes are separated by vertical dotted lines and progenies of individual lines by horizontal lines.

FIG. 12 indicates the role of OsDOT1 in vascular patterning of the rice leaf. Images taken from transverse sections show Leaf 3 middle blade—Osdot1-m2 (F3) grown on 0.1 μM Naphthaleneacetic acid (NAA).

FIG. 13 shows phylogenetic relationships between various orthologues of ZmDOT1 genes in representative plant species. * denotes the presence of an EAR domain.

FIG. 14 shows CRISPR/Cas9-mediated gene editing of the Setaria virisis DOT1. (A) Gene diagram showing the localization (highlighted purple regions) and orientation (purple arrows) of the two sgRNAs used. The PAM motifs are marked in purple italic. (B, C) Cross-sections showing venation patterns in leaf 2 of a young T1 Setaria plant homozygous for the Svdot1-m1 allele (B) or heterozygous (C). M—midvein, L—lateral vein, red arrowhead marks new lateral veins present in the homozygous line. Images were taken at the same scale.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

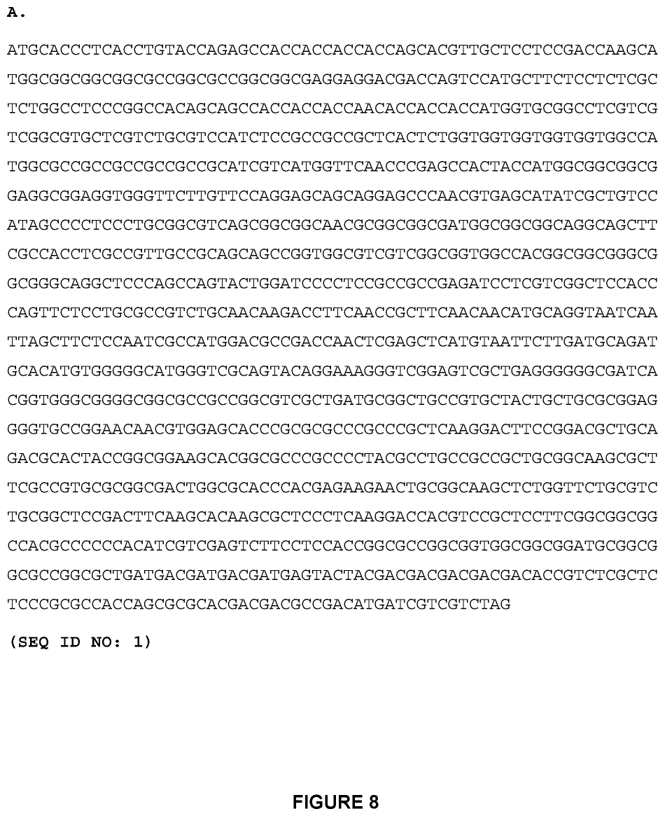

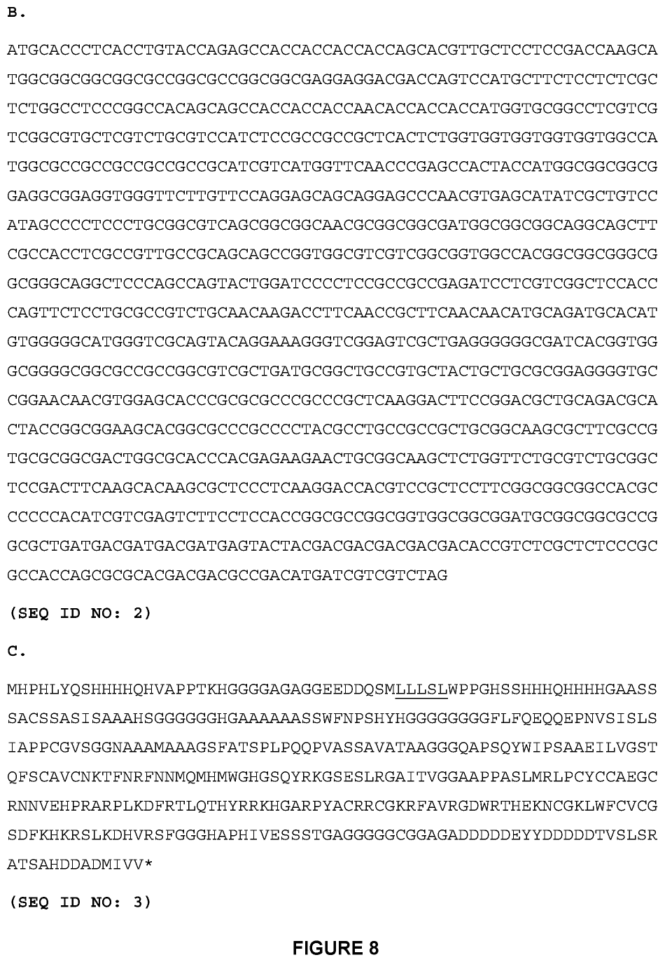

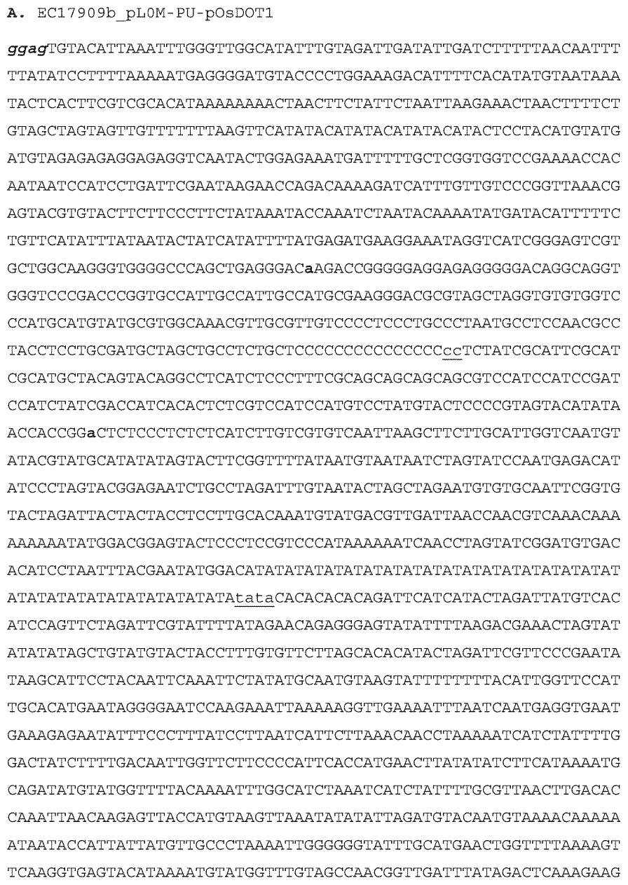

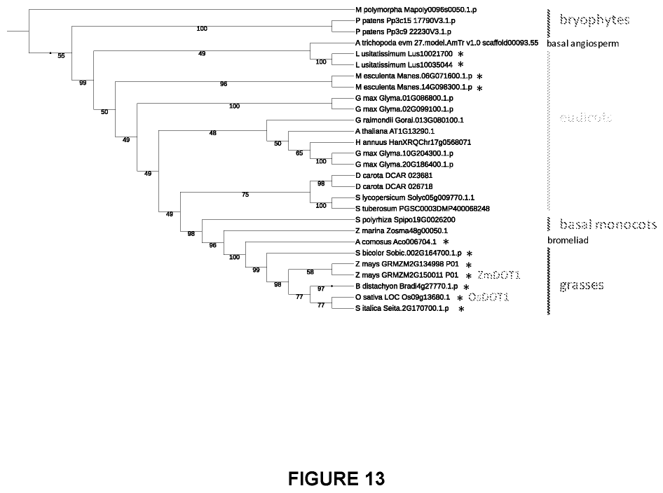

The Zea mays DISORGANIZED TRIBUTARIES 1 (ZmDOT1) gene encodes a zinc finger C2H2 type family protein (see, for example NCBI gene accession GRMZM2G150011; NCBI Reference Sequence: NC_024465.2) (see, for example, Fouracre et al. 2014, J Exp Bot., 65(13):3327-39). Orthologues of ZmDOT1 exist in numerous plant species including, for example, bryophytes, eudicots, basal monocots and grasses (see FIG. 13) for representative plant species and their phylogenetic relationships. A representative orthologue in the grasses (Poaceae/Gramineae) family is the rice gene OsDOT1 (LOC_Os09g13680) (see representative sequences in FIG. 8). The ZmDOT1 and OsDOT1 genes and their orthologues in other plants are, for convenience, collectively referred to herein as the “OsDOT1 orthogroup”. Without limitation, members of the OsDOT1 orthogroup may be identified using routine methods known in the art including, for example, orthofinder software as described in Emms & Kelly, 2019, “OrthoFinder: phylogenetic orthology inference for comparative genomics”, available on GitHub (https://github.com/davidemms/OrthoFinder).

The present inventors have demonstrated that inhibition or loss of function of genes in the OsDOT1 orthogroup provides a means of increasing any one or more of bundle sheath cell numbers, vein density, and/or the proportion of lateral veins. Without being limited by theory, the inventors hypothesise that genes of the OsDOT1 orthogroup encode transcription factors which act in developing veins to laterally inhibit procambium initiation in neighbouring cells. The time and extent to which the encoded proteins function in each vein may thus determine how closely veins are spaced. The phenotypic characteristics arising from inhibiting or preventing the expression of genes within the OsDOT1 orthogroup and/or the activity of their encoded proteins in plants (e.g. plant leaves) can provide increased photosynthetic efficiency. This may arise at least in part from more “C₄-like” photosynthetic activity.

The present invention thus provides genetically modified plant cells in which the expression of genes in the OsDOT1 orthogroup and/or the function of their encoded proteins is suppressed or removed. The modified plants and plant cells described herein can serve as a chassis into which further modifications designed to confer C₄ photosynthetic traits can be introduced.

### OsDOT1 Orthogroup

The skilled person will readily be able identify genes of the OsDOT1 orthogroup, and their associated complementary DNA (cDNA) and protein sequences in a wide variety of plants using standard and non-inventive methods. As known to the skilled addressee there are numerous publicly accessible online tools available which can be used to identify OsDOT1 orthogroup genes and their proteins using input reference sequence/s (e.g. OsDOT1 orthogroup sequences disclosed herein and/or those on publicly available databases such as UniProt, GenBank and the like).

Methods for assessing the level of homology and identity between sequences are well known in the art. The percentage of sequence identity between two sequences may, for example, be calculated using a mathematical algorithm. A non-limiting example of a suitable mathematical algorithm is described in the publication of Karlin and colleagues (1993, PNAS USA, 90:5873-5877). This algorithm is integrated in the BLAST (Basic Local Alignment Search Tool) family of programs (see also Altschul et al. (1990), J. Mol. Biol. 215, 403-410 or Altschul et al. (1997), Nucleic Acids Res, 25:3389-3402) accessible via the National Center for Biotechnology Information (NCBI) website homepage (https://www.ncbi.nlm.nih.gov). The BLAST program is freely accessible at https://blast.ncbi.nlm.nih.gov/Blast.cgi. Other non-limiting examples include the Clustal (http://www.clustal.org/) and FASTA (Pearson (1990), Methods Enzymol. 83, 63-98; Pearson and Lipman (1988), Proc. Natl. Acad. Sci. U.S.A 85, 2444-2448) programs. These and other programs can be used to identify sequences which are at least to some level identical to a given input sequence. Additionally or alternatively, programs available in the Wisconsin Sequence Analysis Package, version 9.1 (Devereux et al. 1984, Nucleic Acids Res., 387-395), for example the programs GAP and BESTFIT, may be used to determine the percentage of sequence identity between two polypeptide sequences. BESTFIT uses the local homology algorithm of Smith and Waterman (1981, J. Mol. Biol. 147, 195-197) and identifies the best single region of similarity between two sequences. Where reference herein is made to an amino acid sequence sharing a specified percentage of sequence identity to a reference amino acid sequence, the difference/s between the sequences may arise partially or completely from conservative amino acid substitution/s. In such cases, the sequence identified with the conservative amino acid substitution/s may substantially or completely retain the same biological activity of the reference sequence.

As noted above, members of the OsDOT1 orthogroup may be identified using routine methods known in the art including, for example, orthofinder software as described in Emms & Kelly, 2019, “OrthoFinder: phylogenetic orthology inference for comparative genomics”, available on GitHub (https://github.com/davidemms/OrthoFinder). Suitable techniques are also described, for example, in Vallender, 2009, “Bioinformatic approaches to identifying orthologs and assessing evolutionary relationships”, Methods; 49(1): 50-55; Altenhoff et al. 2016, “Standardized benchmarking in the quest for orthologs”, Nature Methods, 13(5); 425-433).

While the sequence characteristics of OsDOT1 orthogroup genes and their proteins may vary between different plant types they are readily identifiable using standard methods known in the art (e.g. bioinformatic approaches). Hence, there is no limitation on the type of plant cells and plants in which the expression of OsDOT1 orthogroup genes and/or the activity of their encoded proteins may be inhibited or prevented in accordance with the present invention.

Plants in which the expression of OsDOT1 orthogroup genes and/or the activity of their encoded proteins may be inhibited or prevented include, but are not limited to, any member of the Aceraceae, Anacardiaceae, Apiaceae, Asteraceae, Betulaceae, Brassicaceae, Buxaceae, Chenopodiaceae/Amaranthaceae, Compositae, Cucurbitaceae, Fabaceae, Fagaceae, Gramineae, Juglandaceae, Lamiaceae, Lauraceae, Leguminosae, Moraceae, Myrtaceae, Oleaceae, Platanaceae, Poaceae, Polygonaceae, Rosaceae, Rutaceae, Salicaceae, Solanaceae, Ulmaceae or Vitaceae. Non-limiting examples of suitable gymnosperms include any member of the Cuppressaceae, Pinaceae, Taxaceae or Taxodiaceae.

As described herein, plants in which the expression of OsDOT1 orthogroup genes and/or the activity of their encoded proteins may be inhibited or prevented may be monocotyledons (monocots).

Alternatively, in some embodiments the plants may be dicotyledons (dicots).

In certain embodiments, plants in which the expression of OsDOT1 orthogroup genes and/or the activity of their encoded proteins may be inhibited or prevented include any type of crop plant, such as for example, a cereal crop plant. The plant may be a C₃ grass. The plant may be a wheat, barley, oat, rye or rice plant. According to a specific embodiment the plant may be a rice plant, and according to a more specific embodiment, the plant may be a rice plant expressing one or more components of the C₄ photosynthetic pathway, optionally all necessary components for a complete C₄ photosynthetic pathway.

Other non-limiting examples of plants in which the expression of OsDOT1 orthogroup genes and/or the activity of their encoded proteins be inhibited or prevented include C₃ photosynthetic plants, C₄ photosynthetic plants, CAM plants, genus Oryza plants (e.g. Oryza sativa, Oryza glaberrima, Oryza sativa L ssp. indica, Oryza sativa L ssp. japonica, Oryza sativa Kitaake), Soy (Glycine max), Cotton (Gossypium hirsutum), Oilseed rape/Cannola (B. napus subsp. napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), Cannabis (Cannabis sativa), sugar beet (Beta vulgaris), oat (Avena sativa), rye (Secale cereale), peanut (Arachis hypogaea), Sunflower (Helianthus annuus), flax (Linum spp.), bean (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), and rubber (Hevea brasiliensis).

The expression of OsDOT1 orthogroup genes and/or the activity of their encoded proteins may be inhibited or prevented in plant cells, including but not limited to those from any of the aforementioned plant types. In some embodiments the plant cells may be from the leaves of such plants. The plant cells may, for example, be ground meristem cell of leaves, procambial initial cells of leaves, vascular sheath cells, bundle sheath cells, mestome sheath cells, or mesophyll cells.

The expression of OsDOT1 orthogroup genes and/or the activity of their encoded proteins may be inhibited or prevented in plant pans, including but not restricted to leaves, stems, roots, tubers, flowers, fruits and seeds, fruit, embryos, or shoots, leaves, stems, roots, stolons, tubers, buds, cuttings or other non-reproductive vegetative material thereof obtained from such plants.

In some embodiments, the methods of the present invention may be used to inhibit or prevent the expression of an OsDOT1 orthogroup gene comprising or consisting of a sequence as defined by SEQ ID NO: 1. In other embodiments, the methods may be used to inhibit or prevent the expression of a DOT1 gene comprising or consisting of a sequence having at least: 70%, 75%, 80%, 85%, 87%, 89%, 90%, 91%, 92%, 93%, 94%, 95%, 96%, 97%, 98%, 99%; sequence identity to the sequence as defined in SEQ ID NO: 1.

In other embodiments, the methods of the present invention may be used to inhibit or prevent the activity of a protein encoded by an OsDOT1 orthogroup gene comprising or consisting of a sequence as defined by SEQ ID NO: 3. In still other embodiments, the methods may be used to inhibit or prevent the activity of a protein encoded by an OsDOT1 orthogroup gene comprising or consisting of a sequence having at least: 70%, 75%, 80%, 85%, 87%, 89%, 90%, 91%, 92%, 93%, 94%, 95%, 96%, 97%, 98%, 99%; sequence identity to the sequence as defined in SEQ ID NO: 3.

### Mutant Cells and Plants

The present invention provides modified plants and plant cells in which the expression of OsDOT1 orthogroup genes and/or the activity of their encoded proteins is inhibited or prevented. Any suitable method known to those of skill in the art may be used for this purpose.

Suitable and non-limiting techniques for inhibiting or preventing the expression of OsDOT1 orthogroup genes are described in: Plant Gene Silencing (Methods and Protocols), Mysore & Senthil-Kumar (Eds), 2015, Humana Press.

In some embodiments, gene editing techniques may be used to modify an OsDOT1 orthogroup gene in the genome of a given plant or plant cell such that its expression is inhibited or prevented. For example, gene editing may be used to modify the genome of a plant or plant cell by way of the insertion, deletion, or replacement of one or more segments of the OsDOT1 orthogroup gene to thereby inhibit or prevent DOT1 expression. Suitable gene editing techniques for this purpose are known to those of skill in the art and include, by way of non-limiting example, the CRISPR (clustered regularly interspersed short palindromic repeats)—Cas9 system, TALENs (Transcription activator-like effector nucleases), and ZFNs (zinc finger nucleases). Suitable and non-limiting gene editing techniques for this purpose are described, for example, in: Gene Editing in Plants, in “Progress in Molecular Biology and Translational Science”, 2017, Vol 149, Weeks and Yang (Eds), Elsevier Inc.; and Song et al., Genome Engineering in Human Cells, in “Methods in Enzymology”, 2014, Chapter 5 Volume 546, 93-118, Elsevier Inc.).

In other embodiments, OsDOT1 orthogroup gene expression can be inhibited or prevented by way of replacing or disrupting the OsDOT1 orthogroup gene of a given plant or plant cell using homologous recombination. For example, homologous recombination may be used to replace the full OsDOT1 orthogroup gene or a portion thereof within the genome of a target plant or plant cell with another sequence, thereby inhibiting or preventing expression of the OsDOT1 orthogroup gene. Suitable and non-limiting techniques involving homologous recombination include those described in: Homologous Recombination and Gene Silencing in Plants, Paszkowski (Ed), 1994, Kluwer Academic Publishers. Additionally or alternatively, prime editing can be used for this purpose (see, for example, Xu, et al., Development of Plant Prime-Editing Systems for Precise Genome Editing”, Plant Communications 1, 100043, May 2020—https://www.sciencedirect.com/science/article/pii/S2590346220300262).

In certain embodiments, inhibitory RNA may be used to inhibit or prevent expression of an OsDOT1 orthogroup gene in the genome of a given plant or plant cell. Non-limiting examples include small/short interfering RNA (siRNA) and microRNA (miRNA) based techniques as described, for example, in Guo et al. 2016, “RNA Silencing in Plants: Mechanisms, Technologies and Applications in Horticultural Crops”. Current Genomics, 17(6): 476-489; Kamthan et al. 2015, “Small RNAs in plants: recent development and application for crop improvement”, Front. Plant Sci.; and RNAi and Plant Gene Function Analysis (Methods and Protocols), Kodama & Komamine (Eds), 2011, Human Press.

In still other embodiments, antagonists may be used to inhibit or prevent the activity of proteins encoded by OsDOT1 orthogroup genes in a plant or plant cell. The antagonists may, for example, bind to a portion of the encoded protein in a manner that prevents it from binding to a promoter sequence. Additionally or alternatively, the antagonists may competitively bind to a promoter sequence thus preventing the encoded protein from doing so. Suitable antagonists for inhibiting or preventing the activity of proteins encoded OsDOT1 orthogroup genes may be designed by protein modelling, the screening of libraries, and various other techniques know to those of skill in the art. Non-limiting examples of suitable antagonists may include peptides, small molecules, and the like.

Modified plants and plant cells in which the expression OsDOT1 orthogroup genes and/or the activity of their encoded proteins is/are inhibited or prevented may be generated using recombinant constructs for delivery of nucleic acids into plant cells.

General guidance on suitable methods can be found, for example, in standard texts such as Green and Joseph. (2012), Molecular cloning: a laboratory manual, fourth edition. Cold Spring Harbor, N.Y.: Cold Spring Harbor Laboratory Press; Ausubel et al. (1987-2016). Current Protocols in Molecular Biology. New York, N.Y., John Wiley & Sons; and ‘Cloning a Specific Gene.’ in Griffiths et al. 1999 Modern Genetic Analysis. New York: W. H. Freeman.

Non-limiting examples of vectors that may be used to generate the recombinant constructs for delivery are well known to those of skill in the art and include, without limitation, plasmids, cosmids, yeast vectors, yeast artificial chromosomes, bacterial artificial chromosomes, P1 artificial chromosomes, plant artificial chromosomes, algal artificial chromosomes, modified viruses (e.g. modified adenoviruses, retroviruses or phages), and mobile genetic elements (e.g. transposons). Techniques for producing recombinant nucleic acids and constructs (e.g. recombinant DNA, recombinant RNA, and the like) including those provided in the form of a vector, are also well known to those skilled in the art.

By way of non-limiting example, nucleic acids may be isolated and the sequence/s of interest cloned or synthesised using standard methods to produce large copy numbers. The sequences can then be inserted into appropriate vectors generating the recombinant constructs. To facilitate expression of the target sequences in the plant cell, the recombinant constructs may be operably linked to a suitable promoter sequence facilitating expression of the target sequence/s in the plant cell of interest, and optionally other regulatory sequence/s for that same purpose. Selectable marker/s may also be incorporated into the recombinant constructs so as to facilitate straightforward selection of transformed plant cells.

The recombinant constructs as described above may be introduced into plants by a variety of techniques as known in the art including, for example, Agrobacterium-mediated transformation, cation or polyethylene glycol treatment of protoplasts, electroporation, microinjection, viral infection, protoplast fusion, microparticle bombardment, abrasion of plant material with DNA-coated particles (such as carborundum), agitation of cell suspensions in solution with microbeads or microparticles coated with the transforming DNA, direct DNA uptake, liposome-mediated DNA uptake, and the like, as also described in a wide range of publicly available texts, such as: “Methods for Plant Molecular Biology” (Weissbach & Weissbach, eds., 1988); Clough, S. J. and Bent, A. F. (1998) “Floral dip: a simplified method for Agrobacterium-mediated transformation of Arabidopsis thaliana” Plant J. 16, 735-743; “Methods in Plant Molecular Biology” (Schuler & Zielinski, eds., 1989); “Plant Molecular Biology Manual” (Gelvin, Schilperoort, Verma, Eds., 1993); and “Methods in Plant Molecular Biology—A Laboratory Manual” (Maliga, Klessig, Cashmore, Gruissem & Varner, Eds., 1994). See also Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein; and Ausubel et al. (Eds) Current Protocols in Molecular Biology, John Wiley & Sons (2000).

In certain embodiments, plants and plant cells used to generate the modified plants and plant cells of the present invention may comprise OsDOT1 orthogroup genes comprising one or more ethylene-responsive element binding factor-associated amphiphilic repression (EAR) domain/s. The EAR domain/s may have a motif sequence defined by, for example, LxLxL (e.g. LLLSL), DLNxxP, or an overlap of LxLxL and DLNxxP. Without limitation to theory, it is hypothesised that the EAR domain/s of OsDOT1 orthogroup genes and their encoded proteins may act as transcriptional repressors that can suppress the response to auxin (e.g. vascularisation), given that EAR domains are present in other auxin-responsive genes.

The EAR domain/s may be removed from or with the OsDOT1 orthogroup genes or components thereof when generating the modified plants and plant cells, such that the modified plants and plant cells (i) no longer comprise OsDOT1 orthogroup genes and any EAR domain/s which they may include, or (ii) comprise OsDOT1 orthogroup genes or component/s thereof from which all or at least some EAR domain/s have been removed. By way of non-limiting example, EAR domain/s may be removed from OsDOT1 orthogroup genes and/or their encoded proteins during generation of any of: modified grass (i.e. Poaceae/Gramineae family) or other monocot plants and plant cells (e.g. rice, wheat, maize, barley, oats, rye, common millet, finger millet, teff, sugar cane and sorghum), modified eudicot plants and plant cells, modified bromeliad plants and plant cells; in accordance with embodiments of the present invention.

In certain embodiments, the modified plants and plant cells may additionally comprise recombinant constructs capable of facilitating the expression of auxin or an analogue thereof (e.g. 4-dichlorophenoxyacetic acid (2,4-D), 1-naphthaleneacetic acid (1-NAA), indole-3-acetic acid [IAA], 2,4-D, 1-NAA). The addition of auxin to modified plants and plant cells of the present invention has been observed to enhance vascularisation (e.g. of leaf tissue). Without being bound by theory it is hypothesised that when the expression of OsDOT1 orthogroup genes and/or the activity of their encoded proteins is inhibited or prevented in the plants and plant cells, there is an associated increase in responsiveness to auxin which enhances vascularisation. The expression of additional auxin or analogues thereof within the modified plants and plant cells thus serves as a means to increase the degree of vascularisation in tissues (e.g. leaves). The modified plants and plant cells engineered to express auxin or an analogue thereof may, prior to having been modified to inhibit or prevent the expression of OsDOT1 orthogroup genes and/or the activity of their encoded proteins, initially have comprised an OsDOT1 orthogroup gene comprising one or more ethylene-responsive element binding factor-associated amphiphilic repression (EAR) domain/s. In such modified plants and plant cells, the EAR domain/s in modified plants and plant cells engineered to express auxin or an analogue thereof may be removed from the OsDOT1 orthogroup genes or their encoded proteins.

The preferred method of transformation may depend upon the plant to be transformed. Agrobacterium vectors are often used to transform dicot species, especially members of the Solanaceae and Brassicaceae. However, Agrobacterium-mediated transformation of monocotyledonous species, including rice and wheat, have been known for some time and are now well established (see, for example, International patent publications WO 97/48814; see also Hiei, Y. et al (1994), Plant J. 6(2):271-282 and international patent publication WO 92/06205). Biolistic bombardment with particles coated with transforming DNA/constructs and silicon fibers coated with transforming DNA are often useful for nuclear transformation. More recently nanoparticle-mediated direct delivery of genetic material into plant cells, including monocot cells, has been developed and overcomes a number of the drawbacks associated with other transformation techniques (such as tissue damage, cytotoxicity, apoptosis, necrosis)—see, for example, Cunningham, F. J. et al (2018) Trends in Biotechnology 36(9) DOI: 10.1016/j.tibtech.2018.03.009.

The recombinant constructs may facilitate transient expression of sequence/s of interest in the plant cell. This may result in transient expression of the recombinant sequence/s for a finite period (e.g. 1, 2, 3, 4, 5, 7, 8, 9, or 10 days). Methods for achieving transient expression of recombinant nucleic acids in host cells are well known in the art. In some embodiments, transient expression may be characterised by a lack of replication of the recombinant nucleic acid sequence when the host cell replicates. In some embodiments, transient expression may be characterised by an absence of integration of the recombinant nucleic acid sequence into the genome of the host cell.

Additionally or alternatively, the recombinant constructs may facilitate stable expression of sequence/s of interest in the plant cell. Recombinant nucleic acid sequences that have been stably introduced into the cell will generally be replicated when the host cell replicates.

Any of the methods of the present invention, as discussed above or below, can be used to transform any plant cell. In this manner, genetically modified plants, plant cells, plant tissue, seed, and the like can be obtained.

Plant cells which have been transformed may be grown into plants in accordance with conventional methods as are known in the art (See, for example, McCormick, S. et al (1986), Plant Cell Reports 5:81-84; Gamborg and Phillips, 1995, Plant cell, tissue and organ culture: fundamental methods. Springer, Berlin; Low et al. 2018, ‘Transgenic Plants: Gene Constructs, Vector and Transformation Method’ in New Visions in Plant Science, Çelik (Ed), IntechOpen; Transgenic Crop Plants, Volume 1. Principles and Development, 2010, Kole, Michler, Abbott, Hall, (Eds.); and Methods in molecular biology, volume 286. Transgenic plants. Methods and protocols. L Peña, editor. ed. 2005. Totowa, N.J.: Humana Press). Such cells may be screened early on by their ability to grow in the presence of antibiotics, by virtue of including an antibiotic resistance gene in the recombinant construct (under the regulation of an appropriate promoter). The resulting plants may be self-pollinated, pollinated with the same transformed strain or different strains or hybridised, and the resulting plant(s) expressing the target gene(s) in plant cells identified. Such plants may be identified by virtue of expression of a reporter gene, such as GUS or GFP, in the plant cells. Two or more generations may be grown to ensure that the desired phenotypic characteristic is stably maintained. Alternatively, in vegetatively propagated crops, mature mutant/transgenic plants may be propagated by cutting or by tissue culture techniques to produce identical plants. Selection of mutant/transgenic plants can be carried out and new varieties may be obtained and propagated vegetatively for commercial use. For a general description of plant transformation and regeneration see also, for example, Walbot et al. (1983) in “Genetic Engineering of Plants”, Kosuge et al. (Eds.) Plenum Publishing Corporation, 1983 and “Plant Cell, Tissue and Organ Culture: Fundamental Methods”, Gamborg and Phillips (Eds.), Springer-Verlag, Berlin (1995). See also Sambrook, J. et al., Molecular Cloning, Cold Spring Harbor Laboratory (1989), Sambrook, J. and Russell, D. W. (2001), “Molecular Cloning: A Laboratory Manual”, 3rd edition, Cold Spring Harbor Laboratory Press, and references cited therein; and Ausubel et al. (Eds) Current Protocols in Molecular Biology, John Wiley & Sons (2000).

Plants transformed/mutated by the methods of the present invention may also be screened based on expression of one or more of the inserted gene/s, for example by detecting expression of the introduced nucleotide sequence/s, by molecular analysis using specific oligonucleotide probes and/or amplification of the target gene.

In some embodiments, plants transformed/mutated by the methods of the present invention may be backcrossed with standard/wild-type lines using conventional plant breeding methods in order to combine traits of the standard/wild-type plant and the modified plant into a single line. In certain embodiments the offspring may be repeatedly crossed back to the standard/wild-type plant line to obtain a high yielding mutant plant line (e.g. a crop plant such as rice) exhibiting the mutant phenotype of interest.

In certain embodiments, plants transformed/mutated by the methods of the present invention may be monocotyledonous. In other embodiments, the plants may be dicotyledonous. In still other embodiments, the plants may be C₃ photosynthetic plants, C₄ photosynthetic plants, CAM plants, genus Oryza plants (e.g. Oryza sativa, Oryza glaberrima, Oryza sativa L ssp. indica, Oryza sativa L ssp. japonica, Oryza sativa Kitaake), Soy (Glycine max), Cotton (Gossypium hirsutum), Oilseed rape/Cannola (B. napus subsp. napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), Cannabis (Cannabis sativa), sugar beet (Beta vulgaris), oat (Avena sativa), rye (Secale cereale), peanut (Arachis hypogaea), Sunflower (Helianthus annuus), flax (Linum spp.), bean (Phaseolus vulgaris), lima bean (Phaseolus lunatus), mung bean (Phaseolus mung). Adzuki bean (Phaseolus angularis), Chickpea (Cicer arietinum), tobacco (Nicotiana tabacum), buckwheat (Fagopyrum esculentum), oil palm (Elaeis guineensis), and rubber (Hevea brasiliensis).

Also provided are seeds obtained from transformed/mutated plants of the present invention.

### EXAMPLES

The present invention will now be described with reference to specific Example(s), which should not be construed as in any way limiting.

### Example One: Mutant Osdot1 Alleles

### Materials and Methods

CRISPR Target Design

A Golden Gate CRISPR vector was used to target the OsDOT1 gene. Loss of function transgenic lines were obtained with three independent guide RNAs listed in Table 1. Guides were designed using the online guide RNA selection tool, CRISPOR (Concordet and Haeussler, 2018). The target sequences sgRNA1 and sgRNA3 were designed from the first exon of OsDOT1 and sgRNA4 was designed to hit the genomic locus after the stop codon. Guide RNAs were cloned into Golden Gate (Weber et al. 2011) Level1 gRNA expression cassettes under the control of the OsU3 promoter. TGGC and AAAC overhangs were added to the 5′ termini of the complementary oligonucleotides. The Cas9p gene (Ma et al., 2015) was expressed under the control of the maize ubiquitin promoter and the selection module contained a hygromycin resistance gene driven by the rice actin 1 promoter. In the first CRISPR experiment a single guide RNA (sgRNA1) was expressed whereas the second CRISPR experiment expressed all three.

TABLE 1
Sequence and position of the guides used to
generate loss of function OsDOT1 plants

| | Position | | |
|---|---|---|---|
| | from | | |
| Guide | ATG | sequence | orientation |
| sgRNAI | +9 bp | TTCAACCCGAGCCACTACCA [symbol] | forward |
| | | (SEQ ID NO: 4) | |
| SgRNA3 | +278 bp | TGGTGGCTCTGGTACAGGTG [symbol] | reverse |
| | | (SEQ ID NO: 5) | |
| SgRNA4 | +1305 bp | CGTACAGTCTAGCTAGGTAT [symbol] | forward |
| | | (SEQ ID NO: 6) | |

Plant Transformation

Kitaake rice (O. sativa ssp. japonica) callus transformation and seedling regeneration were performed at 32° C. using a protocol modified from Toki et al., 2006, that can be downloaded at:

https://langdalelab.files.wordpress.com/2015/07/kitaake_transformation_2015.pdf.

Regenerated T0 plantlets resistant to hygromycin were tested by polymerase chain reaction (PCR) to validate the presence of the selection gene HYG (forward primer: 5′-CAACCAAGCTCTGATAGAGT-3′ (SEQ ID NO: 31); reverse primer: 5′-GAAGAATCTCGTGCTTTCA-3′ (SEQ ID NO: 32)) Transgenic plants were transferred to soil (John Innes Compost No. 2) and checked for the presence of induced mutations around the guide RNA target sites.

Rice Germination and Growth Conditions

Twenty seeds per line were treated with CruiserMaxx (CM) fungicide (50 μl CruiserMaxx, 1 ml water). Seeds were dried, placed in petri dishes on water-saturated paper towels and germinated with a 16 h/8 h photoperiod, 30° C. day/25° C. night temperatures. After germination seedlings were transferred to falcon tubes containing ½ MS medium. Two week old seedlings were transferred to soil (John Innes Compost No. 2) and grown under the same conditions described above.

Exogenous Auxin Treatments

For auxin treatments, sterilized seeds were germinated and grown on half concentration Murashige and Skoog medium (½ MS medium) supplemented with 0.1 μM NAA. NAA stocks (100 μM) were prepared in DMSO. An equivalent amount of DMSO was added to the control plates.

Fixation and Embedding of Rice Leaf Samples

Leaf segments (2-3 mm) spanning the whole leaf width were cut from the widest part of the fully expanded flag −1 leaf for each T2 plant analysed. Leaf segments were fixed in 3:1 ethanol:acetic acid for 30 minutes at room temperature and transferred to 70% ethanol. Fixed leaf sections were transferred to tissue embedding cassettes then dehydrated and wax infiltrated in a Tissue Tek Vacuum infiltration Processor (VIP). Sections were then removed from their cassettes, placed in paraffin wax blocks and let to set at 4 degrees C. overnight. Wax blocks were trimmed and 10 μm transverse sections were obtained using a Leica RM2135 rotary microtome. Sections were dried onto microscope slides at 37 degrees C. overnight.

Imaging of Leaf Fresh Sections

To analyse the effects of exogenous auxin on mutant plants, thin free hand sections were executed with a feather blade through the centre of the restricted region in the middle of leaf three that lacked chlorophyll, plus regions situated immediately above and below it. Sections were imaged under UV illumination.

Staining of Leaf Sections

Leaf sections were dewaxed and partially rehydrated prior to staining for 30 minutes with Safranin O (1% in 50% EtOH). The slides were then stained for 1 second with Fast Green (0.5% in 95% EtOH), dehydrated, drained and mounted using DPX.

Imaging and Data Collection

The leaf sections were viewed with a light microscope (Leica DMRB microscope) and imaged at ×20 magnification. Images were used to estimate leaf width, vein density, total bundle sheath cell numbers and quantification of each vein type (lateral, rank1 intermediates and rank 2 intermediates).

Phylogenetic Analysis

Protein sequences of 379 ZmDOT1 orthologues from 52 species were retrieved form Phytozome v12.1 (https://phytozome.jgi.doe.gov/pz/portal.html) using Orthofinder software (Emms and Kelly, 2015). From this list, a subset of 19 species was selected that represented a range of flowering plant taxa, and two bryophyte species were selected as representatives of the earliest divergent land plants. A total of 147 sequences from the 21 species were aligned using MergeAlign (Collingridge and Kelly, 2012). The resulting alignment was trimmed using MEG A (Kumar et al., 2018) to include just the EAR domain and the four zinc finger domains. The best-fitting model parameters (JTTDCMut+I+G4) were estimated and consensus phylogenetic trees were run using Maximum Likelihood from 1000 bootstrap replicates (Nguyen et al., 2015). The OsDOT1 clade was extracted from the tree using the ITOL (Interactive Tree of Life) (Letunic and Bork, 2016) web interface.

Leaf Photosynthetic Traits of Osdot1 Mutants

### Plant Growth Conditions

Plants were grown in a controlled environment growth chamber, under current ambient CO₂ level, at the School of Biological Sciences of Washington State University, Pullman, Wash. (USA). All plants were individually grown in 4-L free drainage pots; soil, irrigation and fertilization were as in Giuliani et al. (2013).

The daily photoperiod was 14 h and light was supplied in a bell-shaped pattern, with a maximum photosynthetic photon flux density (PPFD) of 600 μmol photons m⁻² s⁻¹ incident on the plant canopy for 10 h. Air temperature was set at 22° C. in the dark period; after switching on the light, air temperature tracked the PPFD pattern, with a maximum of 26° C. for 10 h. Air relative humidity was maintained at ˜70%, corresponding to a maximum air Vapor Pressure Deficit (VPD) of ˜1.6 kPa.

### Leaf Physiological Analysis

Measurements of leaf-atmosphere CO₂ and H₂O, and stable carbon isotope exchange were performed in Pullman, Wash. on four-to-five week-old rice plants. The measuring apparatus was composed of a LI-6800 equipment (LI-COR Biosciences, NE, USA) operating as an open system and coupled to a tunable-diode laser absorption spectroscope, which detected ¹²CO₂ and ¹³CO₂ isotopologues (TDLAS model TGA200A, Campbell Scientific, Inc., Logan, Utah, USA). The LI-6800 equipment was equipped with a circular leaf chamber (6800-01A) hosting 6 cm² leaf surface area.

For five rice plants of each genotype two fully expanded leaves were selected on the same stem (excluding the first six leaves from the canopy bottom), and the mid to distal blade portions were used for leaf photosynthetic analysis (n=5). Measurements were taken under atmospheric O₂ molar fraction of 20 mmol mol⁻¹ air (2% O₂) and CO₂ (Cₐ) molar fraction of 400 μmol mol⁻¹ air; PPFD was set at 1500 μmol photons m⁻² s⁻¹, leaf temperature at 30° C. and leaf-to-air VPD was kept at 1.2-1.3 kPa.

The leaf portions were acclimated for about ˜30 min in the leaf chamber, and data were recorded for ˜30-40 min. The rates of net CO₂ assimilation and transpiration per unit (one side) leaf surface area (A, μmol CO₂ m⁻² s⁻¹ and E, mmol m⁻² s⁻¹, respectively), intercellular CO₂ molar fraction (Cᵢ, μmol mol⁻¹ air), and stomatal diffusion conductance to water vapor and CO₂ (g_{s_H2O} and g_{s_CO2}, respectively; mol m⁻² s⁻¹) were determined. Based on the analysis of leaf-atmosphere CO₂ and ¹³CO₂ exchange data, mesophyll diffusion conductance to CO₂ (gₘ, mol CO₂ m⁻² s⁻¹) was estimated according to Evans and von Caemmerer, 2013. The ratios of Cᵢ/Cₐ, A/E, A/g_{s_H2O}, and gₘ/g_{s_CO2} were calculated.

To calculate leaf mass per area (LMA) for each genotype, leaf samples of known surface area were taken from the blade portions used for measurements (n=4). The tissue samples were dried in a ventilated oven at 55° C. until constant weight, and the leaf mass per area (LMA, g m⁻²) was computed.

### Results

### Osdot1 Knock-Out Alleles

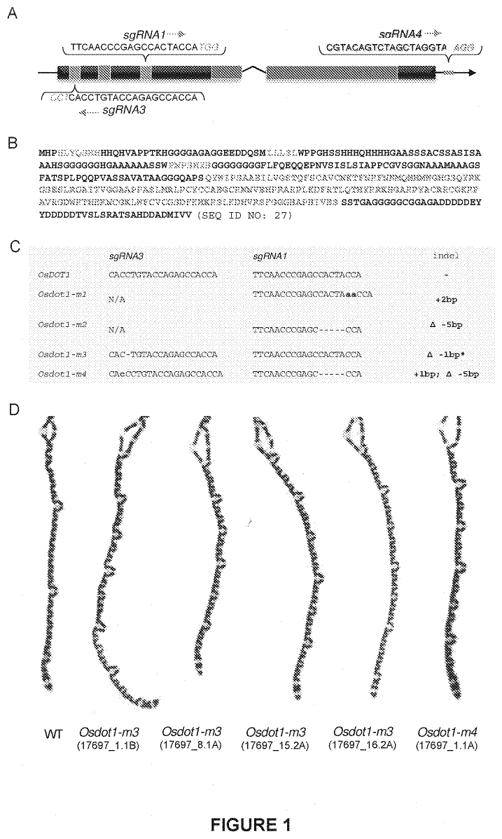

Two recessive knock-out alleles of the rice gene OsDOT1, LOC_Os09g13680, encoding for a putative zinc finger protein (C2H2 family, type IIIA, subclass A1d) were generated using CRISPR in the Kitaake rice background (FIG. 1). In the first allele (m1), a 2 bp insertion results in a premature stop codon and a truncated protein lacking the entire C2H2 zinc-finger domain. The second allele (m2) is characterized by a 5 bp deletion leading to a shift in the reading frame and an altered protein. Both alleles were isolated from the same heteroallelic T0 line.

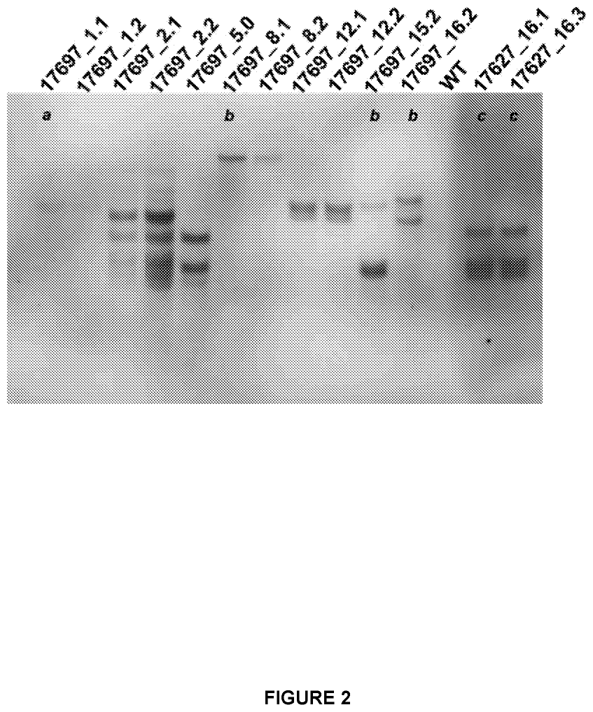

Four new independent knock-out lines carrying alleles m3 and m4 were isolated from a second CRISPR experiment (FIG. 1). The m3 allele was repeatedly isolated from 4 independent transformation events (FIG. 2). The short guide RNAs (sgRNAs) used in both CRISPR experiments are shown in FIG. 1A. In contrast with the first experiment which only employed sgRNA1, the new CRISPR construct used all three sgRNAs aiming to generate a range of mutations affecting the DOT1 gene at positions around the EAR domain (FIG. 1B) including large genomic deletions. The four different alleles obtained in both experiments are summarised in FIG. 1C. Except for allele m1 (truncated) which retains an intact EAR domain, the introduced mutations result in altered or deleted EAR domains.

Vein Morphology in Osdot1 Mutants

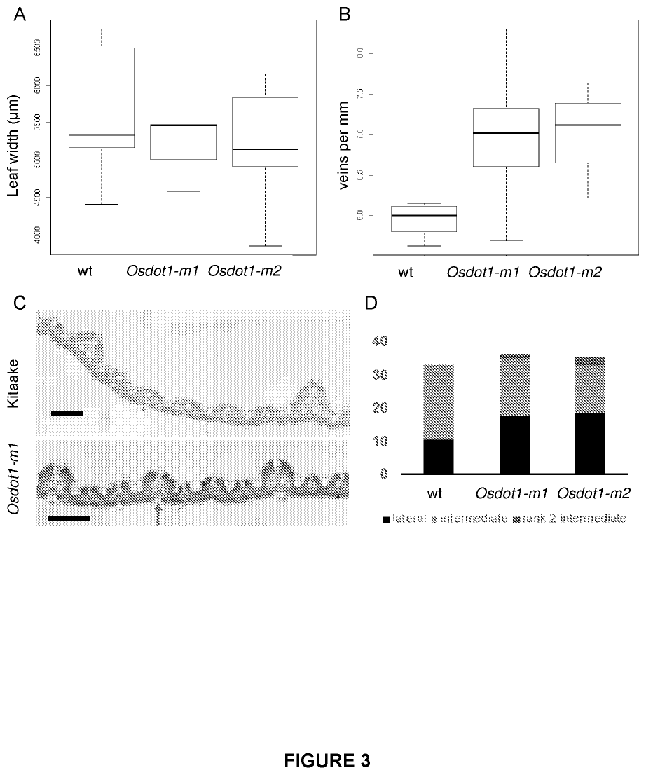

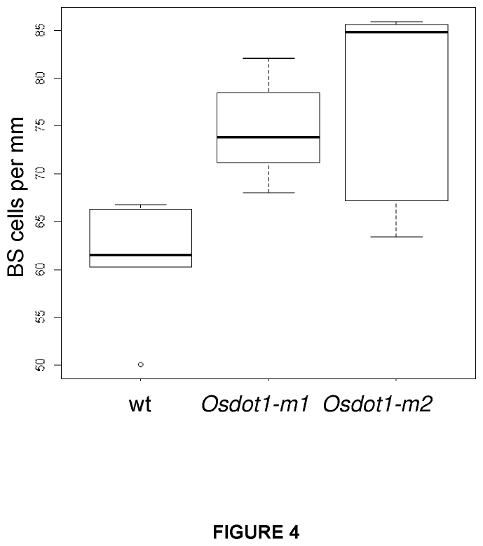

Seedlings carrying alleles m1 and m2 in a homozygous state are characterized by higher vein density and an increased proportion of lateral veins when compared to wild type (FIG. 3A-C). Higher numbers of lateral veins contribute to a greater bundle sheath to mesophyll cell ratio (FIG. 4). Interestingly, the mutant lines also developed some rank 2* intermediate veins not normally found in rice (FIG. 3D).

Although not phenotyped in detail, lines carrying alleles m3 and m4 also consistently show higher numbers of lateral veins when compared to wild type (FIG. 1D). Specific results are summarised below:
Leaf width is not affected in Osdot1 mutants
Mutant alleles can cause increased vein density
Increased numbers of lateral veins are present in the mutant background
Significantly more bundle sheath cells (in contact with the mesophyll) are found in the leaves of mutant plants
A novel type of vein, the rank 2 intermediate (found in C₄ but not C₃ grasses), develops in the Osdot1 background.

*Vein nomenclature

1. Midrib and lateral veins.
Develop acropetally.
Have two large metaxylem elements plus protoxylem or protoxylem lacuna.
Have metaphloem and protophloem when young but at maturity protophloem is crushed.
BS cells are present at left and right of vascular bundle only.
Sclerenchyma is present on both adaxial and abaxial sides connecting the vascular bundle to the epidermis.
2. Intermediate Rank 1.
Develop basipetally.
Have metaxylem (not large) but no protoxylem.
Have metaphloem and may have protophloem.
BS cells encircle the entire vascular bundle.
Slerenchyma may be present between the BS cells and the epidermis on the adaxial, abaxial or both sides.

Osdot1 Mutant Plant Growth

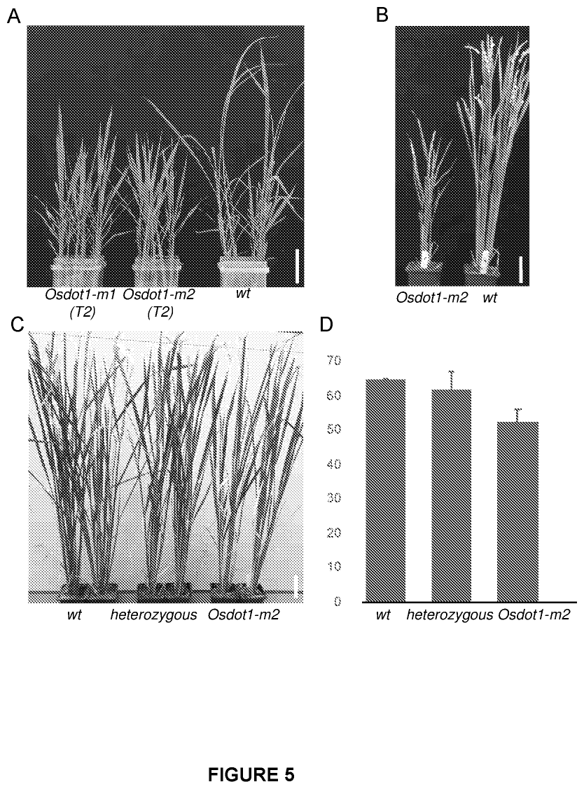

Homozygous mutant plants for m1 and m2 alleles were associated with a reduced plant height phenotype from seedling stage (FIG. 5A) through to maturity (FIG. 5B) in the T2 generation. However, plant height in the homozygous F2 progeny resulting from the backcross of the m2 allele into wild type Kitaake background shows restored plant height (FIG. 5C-D).

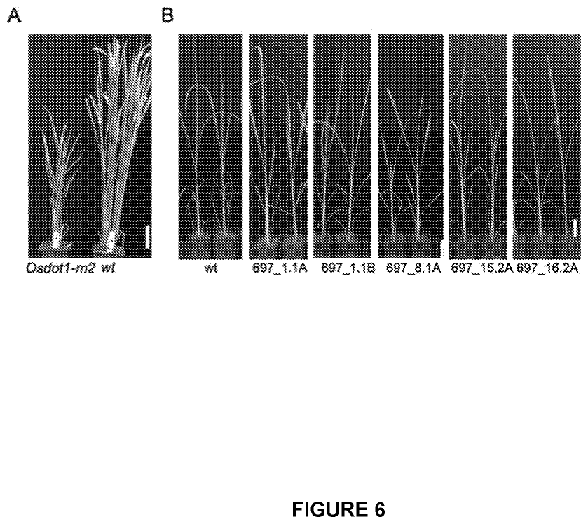

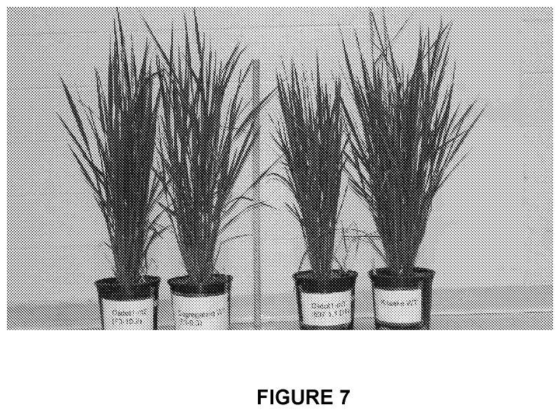

Four independent knock-out lines carrying alleles m3 and m4 isolated from a second CRISPR experiment showed the expected vein patterning phenotype (FIG. 1) but displayed no reduction in plant height (FIG. 6). Furthermore, both m2 and m3 mutant plants appeared to behave in a metabolically similar manner to wild type plants as shown by leaf net CO₂ assimilation and transpiration rates (see Tables 2 and 3, FIG. 7).

Table 2 shows net photosynthetic rate (A), transpiration rate (E), stomatal diffusion conductance to water vapor (g_{s_H2O}) and CO₂ (g_{s_CO2}), and mesophyll diffusion conductance to CO₂ (gₘ). Cᵢ is the intercellular CO₂ molar fraction. Values are mean±SE (n=5). Table 3 shows leaf mass per area (LMA) in WT vs mutant plants. Values are mean±SE (n=4). Lines F3-10.2 and F3-9.3 originate from the backcross of allele m2 into wild type Kitaake background shown in FIG. 3. The Osdot1-m3 (697-1.1D-1) line is the progeny of T1 line 17697_1.1D sister line to 17697_1.1A and 17697_1.1B presented in FIG. 4.

TABLE 2

| | | | | | | | A/E | [symbol] | |
|---|---|---|---|---|---|---|---|---|---|
| | A | E | [symbol] | [symbol] | [symbol] | | (μmol | (μmol | |
| | (μmol | (mmol | (μmol | (mol | (mol | | CO₃ | CO₃ | |
| | CO₂ | H₂O | CO₂ | H₂O | CO₂ | | mol⁻¹ | mol⁻¹ | |
| Plant type | m⁻² s⁻¹) | [symbol] | [symbol] | m⁻² s⁻¹) | m⁻² s⁻¹) | [symbol] | H₂O) | H₂O) | [symbol] |
| Osdot1-m2 | 41.0 ± 1.0 | 5.6 ± 0.2 | 229 ± 3 | 0.44 ± 0.01 | 0.96 ± 0.07 | 0.57 ± 0.01 | 7378 ± 175 | 94.2 ± 1.7 | 3.51 ± 0.33 |
| (F3-10.2) | | | | | | | | | |
| Segregating | 42.1 ± 0.5 | 5.8 ± 0.2 | 230 ± 4 | 0.46 ± 0.02 | 0.96 ± 0.12 | 0.58 ± 0.01 | 7401 ± 165 | 93.3 ± 2.7 | 3.34 ± 0.42 |
| WT (F3-3.3) | | | | | | | | | |
| Osdot1-m3 | 39.6 ± 1.4 | 5.7 ± 04 | 236 ± 3 | 0.44 ± 0.02 | 0.76 ± 0.10 | 0.58 ± 0.01 | 7050 ± 248 | 90.3 ± 2.0 | 2.72 ± 0.28 |
| (697-1.1-D1) | | | | | | | | | |
| WT | 41.6 ± 1.5 | 5.8 ± 0.4 | 241 ± 8 | 0.49 ± 0.04 | 0.82 ± 0.05 | 0.80 ± 0.02 | 7102 ± 358 | 96.9 ± 5.3 | 2.73 ± 0.21 |

[symbol]

indicates data missing or illegible when filed

TABLE 3

| Plant type | LMA (g m⁻²) |
|---|---|
| Osdot1-m2 (F3-10.2) | 67.6 ± 4.7 |
| Segregating WT (F3-9.3) | 61.1 ± 2.4 |
| Osdot1-m3 (697-1.1-D1) | 69.6 ± 6.6 |
| WT | 53.1 ± 5.7 |

Taken together these results suggest that there are no growth or metabolic defects caused by the change of vein patterning in Osdot1 mutants.

Osdot1 Mutant Response to Auxin

Experiments were carried out to test the effect of exogenous auxin on the phenotype of loss of function Osdot1 mutants, specifically Osdot1-m2.

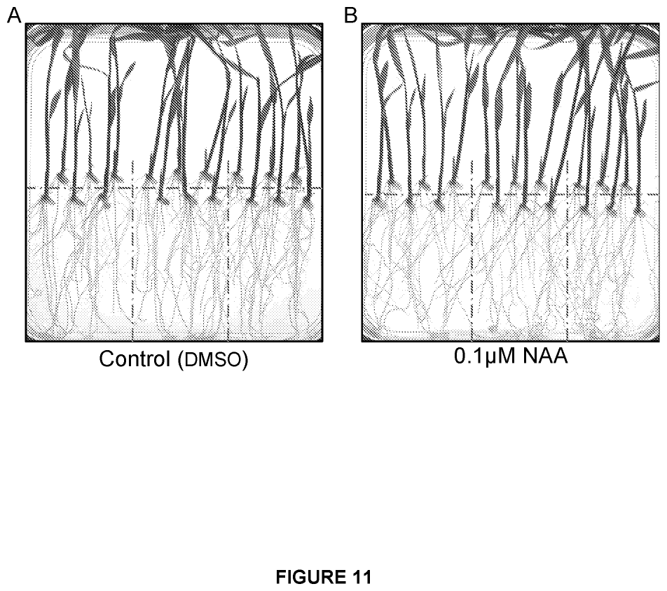

Seeds were plated out on ½ MS medium containing 0.1 μM NAA, grown for 7 days in a growth chamber and then photographed. No overall morphological differences were seen between wild type and mutant seedlings in terms of shoot height, root structure etc. (FIG. 11).

In contrast to wild type, however, mutant plants exhibited a restricted region in the middle of leaf 3. Free hand sections were prepared from this region, plus regions immediately above and below it.

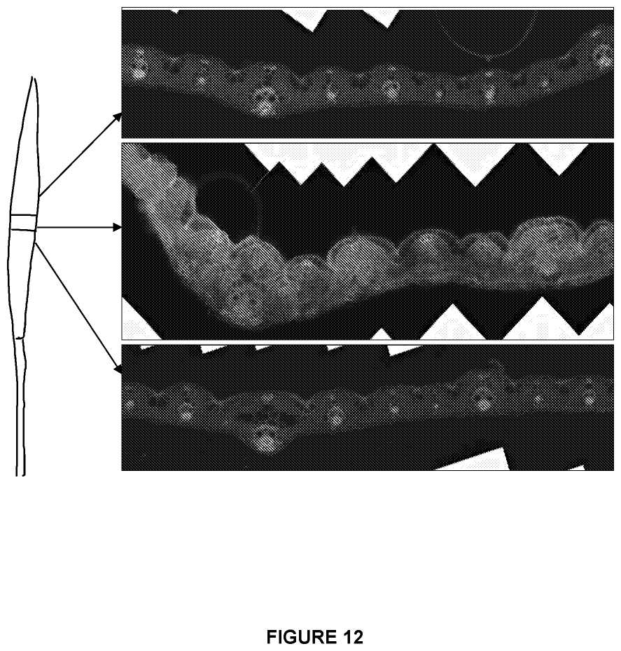

Increased auxin concentrations triggered proliferation of vascular tissue and bulliform cells in the Osdot1-m2 background but not in wild type. The effect, observed on multiple homozygous seedlings, was observed in a narrow region located in middle of the leaf blade on leaf 3 and spanning the entire leaf width (FIG. 12).

### Example Two: miRNA Constructs for Inhibiting OsDOT1 Expression

Artificial miRNAs (amiRNAs)

Artificial microRNA constructs were designed using the oligo designer in WMD (Ossowski et al., 2008). BsaI sites and Golden Gate 4 bp specific overhangs were added to flank the BamHI and KpnI sites already present on the pNW55_osaMIR528 stemloop and subsequently allow the cloning of artificial microRNAs in the Golden Gate system.

The following three microRNAs were designed to target the first exon of LOC_Os09g13680:

(SEQ ID NO: 7)
TTATGCTCACGTTAGGCTCAT
(SEQ ID NO: 8)
TTATGCTCACGTTAGGCTCGT
(SEQ ID NO: 9)
TACAAGAACCCACATCCGCAT

These three microRNAs were used as a basis to design the amiRNA oligonucleotides listed below, destined for the PCR-assisted cloning of the artificial miRNAs into the Golden Gate system:

Oligos for amiRNA “TTATGCTCACGTTAGGCTCAT”

| | | |
|---|---|---|
| I | miR-s | gaTTATGCTCACGTTAGGCTCATtctctcttttgtattcc (SEQ ID NO: 10) |
| II | miR-a | gaATGAGCCTAACGTGAGCATAAtcaaagagaatcaatga (SEQ ID NO: 11) |
| III | miR*s | gaATAAGCCTAACGTCAGCATATtcacaggtcgtgatatg (SEQ ID NO: 12) |
| IV | miR*a | gaATATGCTGACGTTAGGCTTATtctacatatatattcct (SEQ ID NO: 13) |

Oligos for amiRNA “TTATGCTCACGTTAGGCTCGT”

| | | |
|---|---|---|
| I | miR-s | agTTATGCTCACGTTAGGCTCGTcaggagattcagttga (SEQ ID NO: 14) |
| II | miR-a | tgACGAGCCTAACGTGAGCATAActgctgctgctacagcc (SEQ ID NO: 15) |
| III | miR*s | ctACGAGGCTATCGTGAGCATAAtcctgctgctaggctg (SEQ ID NO: 16) |
| IV | miR*a | aaTTATGCTCACGATAGCCTCGTagagaggcaaaagtgaa (SEQ ID NO: 17) |

Oligos for amiRNA “TACAAGAACCCACATCCGCAT”

| | | |
|---|---|---|
| I | miR-s | agTACAAGAACCCACATCCGCATcaggagattcaggttga (SEQ ID NO: 18) |
| II | miR-a | tgATGCGGATGTGGGTTCTTGTActgctgctgctacagcc (SEQ ID NO: 19) |
| III | miR*s | ctATGCGCATGAGGGTTCTTGTAttcctgctgctaggctg (SEQ ID NO: 20) |

### Prophetic:

The artificial microRNA constructs will be cloned and used in rice according to the methods described in (Warthmann et al., 2008), using a Golden Gate compatible version of the backbone vector pNW55. The technology uses the rice endogenous miRNA machinery to generate, in planta, a specific, 21mer small silencing RNA (sRNA) from an integrated transgene. Silencing of the OsDOT1 gene and the subsequent phenotypic change is expected to be observed in the TO generation. To maximize chances of success three different amiRNAs will be cloned and transformed to recapitulate the phenotype observed in the gene-edited lines.

The amiRNA precursor is efficiently processed in rice and abundance of target transcripts are expected to be greatly reduced at least for some of amiRNAs proposed, in T0 lines with high levels of transgene expression. Very low levels of OsDOT1 expression are expected to shift the normal venation pattern towards a Osdot1 mutant phenotype.

### Example Three: Venation Patterns in Plants with Intact and Altered EAR Domains

As described in the Examples above, no obvious difference in venation patterns was observed between plants carrying the m1 allele, with an intact EAR domain, and plants carrying alleles m2, m3 and m4 with an altered EAR domain.

Without being limited to theory, the inventors hypothesize that the well conserved EAR domain in monocots (uniprot data shows a conserved MLLLSLWPPG domain—predicted EAR domain highlighted in bold) might be essential for functional accuracy of DOT1 and a protein lacking the EAR domain might lack normal regulatory feedback.

To test this hypothesis, the present inventors have attempted to express versions of OsDOT1 lacking the EAR domain in its endogenous expression domain.

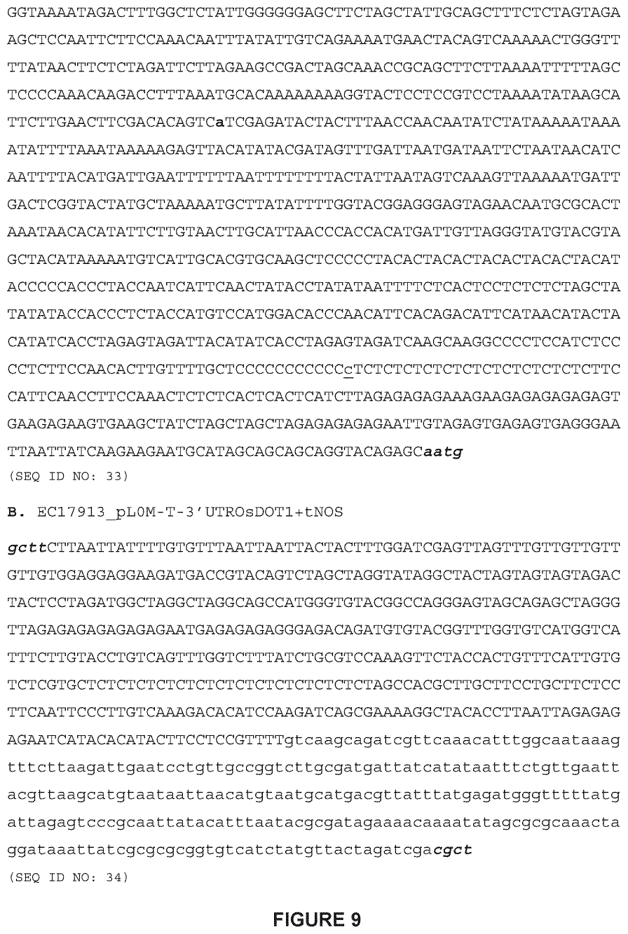

In experiments designed to complement the mutant phenotype, a fragment of 2952 bp upstream from the ATG including the 5′UTR (FIG. 9A) was shown to be insufficient to drive expression of the OsDOT1 cDNA sequence (FIG. 8B). The promoter sequence, modified to be compatible with the Golden Gate cloning system (4 BpiI and BsaI restriction enzyme sites were ‘domesticated’ by introducing a 1 bp change in each case) is presented in (FIG. 9A).

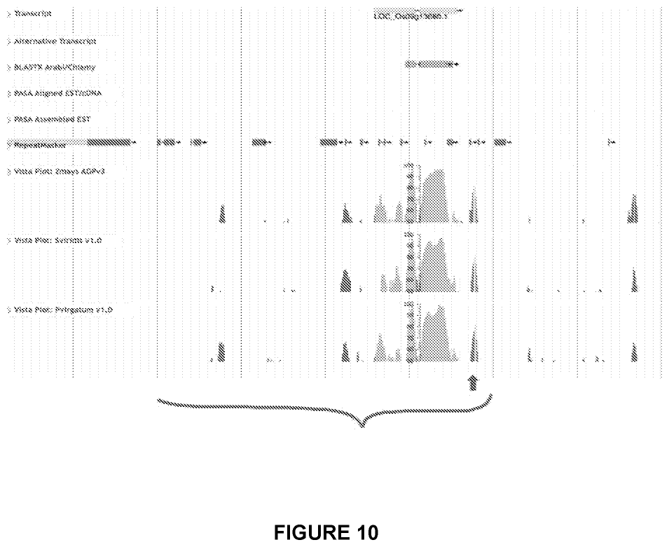

To detect other potentially regulatory sequences, the OsDOT1 locus was aligned with corresponding regions from Z. mays, S. viridis and P. virgatum using the Vista Plot software. The alignment revealed a highly conserved 3′ UTR region (FIG. 10) containing potentially regulatory elements. However, the addition of the conserved 3′UTR region to the complementation construct was not sufficient to drive gene expression from the OsDOT1 promoter shown in FIG. 9A.

Plant introns are often involved in the recruitment of transcription factors leading to either repression or activation of developmental genes, and chromatin signatures, H3K27me and H3K4me, exist in the OsDOT1 intron.

### Prophetic:

The present inventors will (in the future) conduct additional complementation attempts to test the effect of a) the addition of the single intron present in the DOT1 gene and b) the addition of the intron plus a larger (˜5 kb) upstream regulatory region, in the context of a genomic clone.

It is expected that upon successful identification of regulatory sequences that drive transgene expression in the endogenous OsDOT1 expression domain, use of those regulatory sequences to drive transgene expression in a mutant Osdot1 background will determine the relevance of the EAR domain. Specifically, it is expected that expression of a full length OsDOT1 cDNA sequence will lead to complementation of the mutant phenotype whereas expression of a cDNA sequence with a deleted EAR domain will fail to complement the mutant phenotype.

### Example 4: Generating S. viridis (ME034V) CRISPR Lines

### Materials and Methods

### CRISPR Target Design

To generate SvDOT1 mutant alleles, two short guide RNAs (Table 2) targeting the first exon of the gene were designed using CRISPOR. Both guides expressed from the OsU3 promoter were assembled in a single construct, C402006, using the Golden Gate strategy described for rice.

TABLE 4
Sequence and position of the guides used generate
loss of function to SvDOT1 plants plants. Proto-
spacer adjacent motifs (PAM) are shown in bold
italic

| | Posi- | | |
|---|---|---|---|
| | tion | | |
| | from | | orienta- |
| Guide | ATG | sequence | tion |
| sgRNA74 | +61 bp | CTTGTTGCTGGGGGGAACGG [symbol] | reverse |
| | | (SEQ ID NO: 21) | |
| sgRNA153 | +153 bp | AGCATGCCGAGTAGGATGAG [symbol] | reverse |
| | | (SEQ ID NO: 22) | |

### Plant Transformation

Transgenic Setaria viridis ME034V plants were obtained using an Agrobacterium mediated callus transformation method described by Van Eck et al., 2017. Regenerated T0 seedlings with healthy roots were transferred to soil and grown in the greenhouse with 16 h/8 h (Day/night) photoperiod and temperatures of 28° C./22° C. (Day/night).

Recovered seedlings were genotyped for the presence of the transgene using forward primer: 5′-CAACCAAGCTCTGATAGAGT-3′ (SEQ ID NO: 23) and reverse primer: 5′-GAAGAATCTCGTGCTTTCA-3′ (SEQ ID NO: 24) targeting the selection gene, HYG, and checked for editing using commercial Sanger sequencing.

### Growing Setaria

Non-dormant seeds were germinated on damp paper towels in sealed petri-dishes placed in an incubator with 16 hours light at 30° C., 8 hours dark at 24°). When seedlings were big enough to handle they were transferred to soil and grown under greenhouse conditions with 16 h/8 h (Day/night) photoperiod and temperatures of 28° C./22° C. (Day/night)

### Genotyping

DNA suitable for PCR amplification was isolated using a one-step extraction method developed by Kasajima et al., 2004. A 375 bp amplicon was obtained with primers SvDOT1_sg65-F 5′-CACTTGTTTCTCCCCTCCCT-3′ (SEQ ID NO: 25) and SvDOT1_sg153-R 5′-TGTAGTGACTCTGGTGGTGG-3′ (SEQ ID NO: 26) and analysed for eventual mutation by Sanger sequencing.

### Phenotyping

### (i) Fixation and Embedding of Rice Leaf Samples

The tip (4-5 mm) of second emerging leaf was harvested 5 days post germination and fixed in 3:1 ethanol:acetic acid for 30 minutes at room temperature and transferred to 70% ethanol. Fixed leaf sections were transferred to tissue embedding cassettes then dehydrated and wax infiltrated in a Tissue Tek Vacuum Infiltration Processor (VIP). Sections were then placed in paraffin wax blocks, set at 4 degrees C., trimmed and sectioned using a Leica RM2135 rotary microtome. 10 μm transverse sections were dried onto microscope slides

### (ii) Staining of Leaf Sections

Leaf sections were dewaxed and partially rehydrated prior to staining for 30 minutes with Safranin O (1% in 50% EtOH and for 1 second with Fast Green (0.5% in 95% EtOH) then dehydrated and mounted using DPX

(iii) Imaging and Data Collection

The leaf sections were viewed with a light microscope (Leica DMRB microscope) and imaged at ×20 magnification. Images were used to estimate leaf width, vein density, total bundle sheath cell numbers and quantification of each vein type (lateral, rank1 intermediates and rank 2 intermediates).

### Results

### Svdot1 Knock-Out Alleles

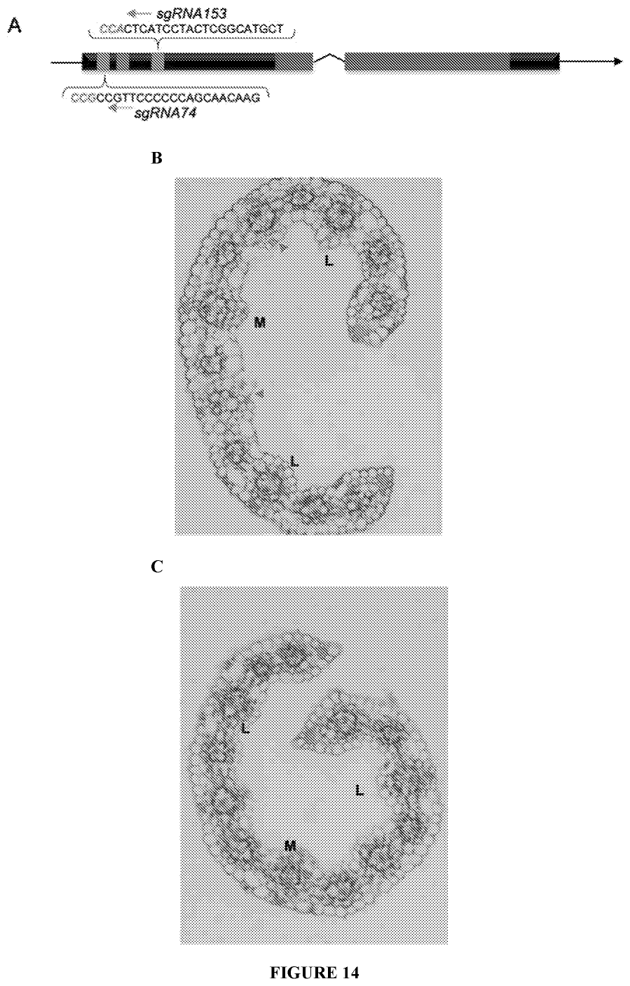

Two independent guides were used to target the first exon of the S. viridis DOT1 gene (FIG. 14A) flanking the conserved EAR domain. Setaria viridis ME034V was successfully transformed using the Golden Gate construct outlined below:

pL2V-pOsAct1-HYG_pZmUbi-Cas9p_sgRNA-OsU3-SvDOT1-g153_sgRNA-OsU3-SvDOT1-g74

Outline of the Golden Gate Construct Used for Setaria Transformation

simultaneously expressing both guides. A total of 11 T0 lines genotyped positive for the presence of the hygromycin gene were isolated from 11 independent regenerating calli in two transformation experiments.

A first mutant allele (m1), identified in the first transformation experiment, has a one bp insertion at sgRNA153 (Table 4) causing a frame-shift and resulting in a partially altered protein starting from amino acid 54, retaining an intact EAR domain but lacking the entire C2H2 zinc-finger domain (as shown in the protein alignment below):

| | | |
|---|---|---|
| | | 1 30 60 90 120 |
| | | \|~~~~~~~~~~~~~~~~~~~~~~~~~~~~♦~~~~~~~~~~~~~~~~~~~~~~~~~~~~~♦~~~~~~~~~~~~~~~~~~~~~~~~~~~~~♦~~~~~~~~~~~~~~~~~~~~~~~~~~~~~\| |
| SEQ ID NO: 28 | SvDOT1 | [symbol] |
| SEQ ID NO: 29 | Svdot1_m1 | [symbol] |
| SEQ ID NO: 30 | Consensus | [symbol] |
| | | 121 150 180 210 240 |
| | | \|~~~~~~~~~~~~~~~~~~~~~~~~~~~~♦~~~~~~~~~~~~~~~~~~~~~~~~~~~~~♦~~~~~~~~~~~~~~~~~~~~~~~~~~~~~♦~~~~~~~~~~~~~~~~~~~~~~~~~~~~~\| |
| | SvDOT1 | [symbol] |
| | Svdot1_m1 | [symbol] |
| | Consensus | [symbol] |
| | | 241 270 300 330 357 |
| | | \|~~~~~~~~~~~~~~~~~~~~~~~~~~~~♦~~~~~~~~~~~~~~~~~~~~~~~~~~~~~♦~~~~~~~~~~~~~~~~~~~~~~~~~~~~~♦~~~~~~~~~~~~~~~~~~~~~~~~~~\| |
| | SvDOT1 | [symbol] |
| | Svdot1_m1 | [symbol] |
| | Consensus | [symbol] |

[symbol]

indicates data missing or illegible when filed

Alignment of the wild type (SvDOT1) and mutant (Svdot1-m1) protein showing frame shift changes introduced by the 1 bp insertion.

A homozygous line for the m1 allele and a heterozygous line were identified in the segregating T1 progeny of line C402006_2.2.

### Vein Morphology in Svdot1 Mutants

Preliminary results show that a T1 5 days old seedling homozygous for allele m1 might result in extra lateral veins when compared to a heterozygous sibling. However, the morphology of fully expanded leaf will have to be assessed to fully validate the mutant phenotype. Both sections in the figure below are taken at equal distance from the tip, 4-5 mm (the whole leaf blade was under 2 cm).

### References Cited in Examples

Concordet, J. P. and Haeussler, M. (2018): CRISPOR: intuitive guide selection for CRISPR/Cas9 genome editing experiments and screens. Nucleic Acids Res, 46(W1): W242-W245.
Collingridge, P. W. and Kelly, S. (2012). MergeAlign: improving multiple sequence alignment performance by dynamic reconstruction of consensus multiple sequence alignments. BMC Bioinformatics 13, 117. doi:10.1186/1471-2105-13-117.
Emms, M. D. and Kelly, S. (2015): OrthoFinder: solving fundamental biases in whole genome comparisons dramatically improves orthogroup inference accuracy. Genome Biology, 16, Article number: 157.
Evans J. R., von Caemmerer S. (2013): Temperature response of carbon isotope discrimination and mesophyll conductance in tobacco. Plant Cell Environ 36:745-756.
Giuliani R., Koteyeva N., Voznesenskaya E., Evans M. A., Cousins A. B., Edwards G. E. (2013): Coordination of leaf photosynthesis, transpiration, and structural traits in rice and wild relatives (Genus Oryza). Plant Physiol 162: 1632-1651.
Kasajima I., Ide Y., Ohkama-Ohtsu N., Yoneyama T., Fujiwara T. (2004) A protocol for rapid DNA extraction from Arabidopsis thaliana for PCR analysis. Plant Mol. Biol. Rep. 22, 49-52.
Kumar, S., Stecher, G., Li, M., Knyaz, C., Tamura, K. (2018): MEGA X: molecular evolutionary genetics analysis across computing platforms. Mol Biol Evol. 35(6):1547-1549.
Letunic, I. and Bork, P. (2016): Interactive tree of life (iTOL) v3: an online tool for the display and annotation of phylogenetic and other trees, Nucleic Adds Research, Volume 44, Issue W1, Pages W242-W245, https://doi.org/10.1093/nar/gkw290.
Nguyen, L-T., Schmidt, H. A., von Haeseler, A., Minh, B. Q. (2015): IQ-TREE: a fast and effective stochastic algorithm for estimating maximum-likelihood phylogenies. Mol Biol Evol. 32(1):268-274.
Ossowski, S., Schwab R., Weigel D. (2008): Gene silencing in plants using artificial microRNAs and other small RNAs, Plant J 53: 674-690.
Petricka, J. J., Clay, N. K., Nelson, T. M. (2008): Vein patterning screens and the defectively organized tributaries mutants in Arabidopsis thaliana. Plant J. 2008; 56(2):251-263. doi:10.1111/j.1365-313X.2008.03595.x.
Sanger F; Coulson A R (1975): A rapid method for determining sequences in DNA by primed synthesis with DNA polymerase. J. Mol. Biol. 94 (3): 441-8. doi:10.1016/0022-2836(75)90213-2. PMID 1100841.
Toki, S., Hara. N., Ono, K., Onodera, H., Tagiri, A., Oka, S., Tanaka, H. (2006): Early infection of scutellum tissue with Agrobacterium allows high-speed transformation of rice. The Plant Journal, 47(6):969-976.
Van Eck, J., Swartwood, K., Pidgeon, K., and Maxson-Stein, K. (2017). Agrobacterium tumefaciens-mediated transformation of Setaria viridis,” in Genetics and Genomics of Setaria, eds A. Doust and X. Diao (Cham: Springer International Publishing), 343-356. doi: 10.1007/978-3-319-45105-3_20.
Warthmann N., Chen H., Ossowski S., Weigel D., Hervé P. (2008): Highly Specific Gene Silencing by Artificial miRNAs in Rice, PLoS ONE 3(3): e1829.
Weber, E., Engler. C., Gruetzner, R., Werner, S., Marillonnet, S. (2011): A modular cloning system for standardized assembly of multigene constructs. PLoS One, 6(2): e16765. https://doi.org/10.1371/journal.pone.0016765.
Xingliang Ma, Qunyu Zhang, Qinlong Zhu, Wei Liu, Yan Chen, Rong Qiu, Bin Wang, Zhongfang Yang, Heying Li, Yuru Lin, Yongyao Xie, Rongxin Shen, Shuifu Chen, Zhi Wang, Yuanling Chen, Jingxin Guo, Letian Chen, Xiucai Zhao, Zhicheng Dong, Yao-Guang Liu. (2015): A Robust CRISPR/Cas9 System for Convenient, High-Efficiency Multiplex Genome Editing in Monocot and Dicot Plants, Molecular Plant. https://doi.org/10.1016/j.molp.2015.04.007.

### INCORPORATION BY CROSS-REFERENCE

The present application claims priority from Australian provisional patent application Nos. 2019902678 and 2019903883, and the entire content of each document is incorporated herein by cross-reference.

1. A plant cell modified to inhibit or prevent expression of a gene as defined in SEQ ID NO: 1 or an orthologue thereof, and/or activity of a protein encoded by the gene, within the cell.

2. The plant cell of claim 1, wherein the plant cell is genetically modified to inhibit or prevent said expression of the gene or orthologue thereof.

3. The plant cell of claim 2, wherein the plant cell:
(i) is genetically modified by knockout of said expression of the gene or orthologue thereof; or
(ii) is genetically modified by knockout of said expression of the gene or orthologue thereof, wherein the knockout of said expression arises from deletion of all or a part of the gene; or
(iii) is genetically modified by knockout of said expression of the gene or orthologue thereof, wherein the knockout of said expression arises from a frameshift mutation, a stop mutation, or a combination thereof, introduced into the gene; or
(iv) is engineered to express inhibitory RNA molecules capable of knocking down expression of the gene in the plant cell.

4. (canceled)

5. (canceled)

6. (canceled)

7. The plant cell of claim 3, wherein:
(i) the inhibitory RNA molecules comprise microRNA or small interfering RNA (siRNA); or
(ii) the plant cell comprises an expression vector for expressing the inhibitory RNA molecules; or
(iii) the plant cell is engineered to express an antagonist capable of inhibiting said activity of the protein encoded by the gene; or
(iv) the plant cell is engineered to express an antagonist capable of inhibiting said activity of the protein encoded by the gene and comprises an expression vector for expressing the antagonist.

8. (canceled)

9. (canceled)

10. (canceled)

11. The plant cell of claim 1, wherein:
(i) the gene comprises or consists of a nucleotide sequence having at least: 70%, 75%, 80%, 85%, 87%, 89%, 90%, 91%, 92%, 93%, 94%, 95%, 96%, 97%, 98%, 99%; sequence identity to a nucleotide sequence as defined in SEQ ID NO: 1; or
(ii) the protein comprises or consists of an amino acid sequence as defined in SEQ ID NO: 3, or having at least: 70%, 75%, 80%, 85%, 87%, 89%, 90%, 91%, 92%, 93%, 94%, 95%, 96%, 97%, 98%, 99%; sequence identity to an amino acid sequence as defined in SEQ ID NO: 3.

12. (canceled)

13. The plant cell of claim 1, wherein the gene and/or protein comprises one or more ethylene-responsive element binding factor-associated amphiphilic repression (EAR) domains, optionally wherein the one or more EAR domains comprise a motif sequence defined by LxLxL (e.g. LLLSL), DLNxxP, or an overlap of LxLxL and DLNxxP.

14. The plant cell of claim 1, wherein the expression of the gene and/or the activity of the protein is inhibited or prevented by deletion of all or a portion of one or more of said EAR domains from the gene and/or protein, or, binding of antagonist/s to the EAR domain/s.

15. The plant cell of claim 13, wherein the plant cell is: (i)
a Poaceae/Gramineae (grass), Linaceae, Euphorbiaceae, or Bromeliaceae; family member; or
(ii) any of a: rice, wheat, maize, barley, oat, rye, common millet, finger millet, teff, sugar cane, or sorghum; cell; or
(iii) any of: Ananas comosus, Manihot esculenta, Setaria italica, Oryza sativa, Oryza sativa japonica, Oryza sativa Kitaake, Brachypodium distchyon, Zea mays, Sorghum bicolor, Anasus comosus, Linum usitatissimum.

16. (canceled)

17. (canceled)

18. The plant cell of claim 13, wherein the plant cell is genetically modified to express auxin or an analogue thereof (e.g. 4-dichlorophenoxyacetic acid (2,4-D), 1-naphthaleneacetic acid (1-NAA), indole-3-acetic acid [IAA], 2,4-D, 1-NAA), optionally comprising an expression vector encoding said auxin or analogue thereof.

19. The plant cell of claim 1, wherein the plant cell is a monocotyledonous plant cell.

20. A method for providing the plant cell of claim 1, the method comprising:
obtaining vectors comprising one or more sequences capable of inhibiting or preventing expression of a gene as defined in SEQ ID NO: 1 or an orthologue thereof, and/or activity of a protein encoded by the gene, within the plant cell;
subjecting a population of plant cells to a transformation procedure using the vectors to thereby provide a subpopulation of plant cells transformed by the vectors; and
selecting a member of said subpopulation of plant cells from said population based on expression of a selectable marker indicative of said transformation, to thereby provide the plant cell.

21. The method of claim 20, wherein the one or more sequences:
(i) encode clustered regularly interspaced short palindromic repeats (CRISPR) guide RNA and a Cas9 nuclease, wherein the guide RNA is specific for the gene and comprises a Protospacer Adjacent Motif (PAM) specific for the Cas9 nuclease; or
(ii) comprise a sequence capable of homologous recombination with the gene of the plant cell to thereby remove all or a portion the gene and thereby render the gene non-functional; or
(iii) encode an antagonist of expression or activity of the protein; or
(iv) encode an antagonist of expression or activity of the protein, wherein the antagonist of the protein expression is an inhibitory RNA molecule; or
(v) encode an antagonist of expression or activity of the protein, wherein the antagonist of the protein activity is a protein capable of binding to the protein to thereby inhibit or prevent said protein activity.

22. (canceled)

23. (canceled)

24. (canceled)

25. (canceled)

26. The method of claim 20, wherein the gene and/or protein comprises one or more EAR domains, and said inhibiting or preventing comprises: removal of all or a part of each said EAR domain from the gene and/or protein, or, binding of antagonist/s to each said EAR domain.

27. The method of claim 26, wherein the plant cell is:
(i) a Poaceae/Gramineae (grass), Linaceae, Euphorbiaceae, or Bromeliaceae; family member;
(ii) any of a: rice, wheat, maize, barley, oat, rye, common millet, finger millet, teff, sugar cane, or sorghum; cell;
(iii) any of: Ananas comosus, Manihot esculenta, Setaria italica, Oryza sativa, Oryza sativa japonica, Oryza sativa Kitaake, Brachypodium distchyon, Zea mays, Sorghum bicolor, Anasus comosus, Linum usitatissimum.

28. The method of claim 20, further comprising genetically engineering the plant cell to express auxin or an analogue thereof (e.g. 4-dichlorophenoxyacetic acid (2,4-D), 1-naphthaleneacetic acid (1-NAA), indole-3-acetic acid [IAA], 2,4-D, 1-NAA).

29. The method of claim 28, comprising transforming the plant cell with an expression vector encoding said auxin or analogue thereof.

30. The method of claim 20, wherein the plant cell is any of: a leaf ground meristem cell, a leaf procambial initial cell, a vascular sheath cell, a bundle sheath cell, a mestome sheath cell, a mesophyll cell.

31. The method of claim 20, wherein the plant cell is C3 photosynthetic plant cell or a C4 photosynthetic plant cell.

32. (canceled)

33. (canceled)

34. (canceled)

35. The plant cell of claim 1, wherein the plant cell is a: genus Oryza plant cell, Soy (Glycine max), Cotton (Gossypium hirsutum), Oilseed rape/Cannola (B. napus subsp. napus), Potato (Solanum tuberosum), tomato (Solanum lycopersicum), Cassava (Manihot esculenta), Wheat (Triticum aestivum), Barley (Hordeum vulgare), pigeon pea (Cajanus cajan), cowpea (Vigna unguiculata), pea (Pisum sativum), Cannabis (Cannabis sativa), sugar beet (Beta vulgaris), oat (Avena sativa), rye (Secale cereale), peanut (Arachis hypogaea), Sunflower (Helianthus annuus), flax (Linum spp.), bean (Phaseolus vulgaris), lima bean (Phaseolus lunatus), mung bean (Phaseolus mung), Adzuki bean (Phaseolus angularis), Chickpea (Cicer arietinum), tobacco (Nicotiana tabacum), buckwheat (Fagopyrum esculentum), oil palm (Elaeis guineensis), Oryza sativa L ssp. indica, Oryza sativa L ssp. japonica, Oryza sativa Kitaake, Oryza sativa japonica, rubber (Hevea brasiliensis), or monocotyledonous; plant cell.

36. A genetically modified plant or a seed thereof comprising the plant cell of claim 1.

37. (canceled)

38. (canceled)

39. (canceled)

40. (canceled)

41. (canceled)

42. (canceled)

43. (canceled)
